(19) United States
(12) Patent Application Publication
(10) Pub. No.: US 2018/0327355 A1
(43) Pub. Date: Nov. 15, 2018

(54) ISOINDOLINE DERIVATIVES

(71) Applicant: VIIV HEALTHCARE UK LIMITED, Brentford, Middlesex (GB)

(72) Inventors: Brian Alvin JOHNS, Research Triangle Park, NC (US); Emile Johann VELTHUISEN, Research Triangle Park, NC (US); Jason Gordon WEATHERHEAD, Research Triangle Park, NC (US)

(21) Appl. No.: 15/776,117

(22) PCT Filed: Dec. 1, 2016
(86) PCT No.: PCT/IB2016/057262

§ 371 Date: May 15, 2018

Related U.S. Application Data
(60) Provisional application No. 62/262,935, filed on Dec. 4, 2015.

(51) Int. Cl. C07D 209/44; C07D 405/06; A61P 31/18

(52) U.S. Cl. CPC C07D 209/44; A61P 31/18; C07D 405/06

(57) ABSTRACT

Compounds of Formula I are disclosed and methods of treating viral infections with compositions comprising such compounds.

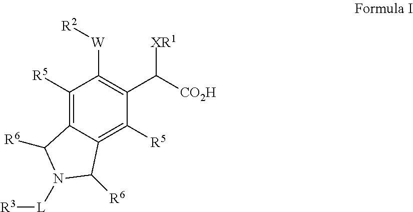

### FIELD OF THE INVENTION

The present invention relates to substituted isoindoline compounds, pharmaceutical compositions, and methods of use thereof for (i) inhibiting HIV replication in a subject infected with HIV, or (ii) treating a subject infected with HIV, by administering such compounds.

### BACKGROUND OF THE INVENTION

Human immunodeficiency virus type 1 (HIV-1) leads to the contraction of acquired immune deficiency disease (AIDS). The number of cases of HIV continues to rise, and currently over twenty-five million individuals worldwide suffer from the virus. Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still required because of undesirable drug-drug interactions; drug-food interactions; non-adherence to therapy; and drug resistance due to mutation of the enzyme target.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur. The emergence of multidrug-resistant HIV-1 isolates has serious clinical consequences and must be suppressed with a new drug regimen, known as salvage therapy.

Current guidelines recommend that salvage therapy includes at least two, and preferably three, fully active drugs. Typically, first-line therapies combine three to four drugs targeting the viral enzymes reverse transcriptase and protease. One option for salvage therapy is to administer different combinations of drugs from the same mechanistic class that remain active against the resistant isolates. However, the options for this approach are often limited, as resistant mutations frequently confer broad cross-resistance to different drugs in the same class. Alternative therapeutic strategies have recently become available with the development of fusion, entry, and integrase inhibitors. However, resistance to all three new drug classes has already been reported both in the lab and in patients. Sustained successful treatment of HIV-1-infected patients with antiretroviral drugs will therefore require the continued development of new and improved drugs with new targets and mechanisms of action.

For example, over the last decade HIV inhibitors have been reported to target the protein-protein interaction between HIV-1 integrase and Lens Epithelium Derived Growth Factor/p75 (“LEDGF”). LEDGF is a cellular transcriptional cofactor of HIV-1 integrase that promotes viral integration of reverse transcribed viral cDNA into the host cell's genome by tethering the preintegration complex to the chromatin. Because of its crucial role in the early steps of HIV replication, the interaction between LEDGF and integrase represents another attractive target for HIV drug therapy.

U.S. provisional patent application 62/027,359 discloses certain isoindoline compounds having the following formula:

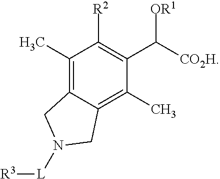

### SUMMARY OF THE INVENTION

Briefly, in one aspect, the present invention discloses compounds of Formula I:

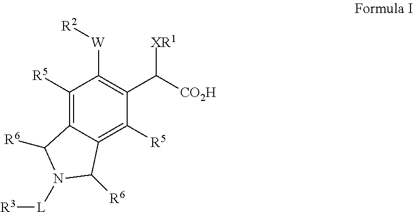

wherein:

X is O or CH₂;

R¹ is C₁₋₆alkyl wherein said alkyl may contain cycloalkyl portions;

W is —CH═CH—, —C≡C—, C₁₋₃alkylene, —CH₂C(O)NH—, —NHC(O)—, —N(CH₃)C(O)—, —N(CH₃)C(O)CH₂—, —C(O)—, —CH₂C(O)—, or —NHC(O)CH₂—, wherein each W is optionally substituted by 1 or 2 methyl groups;

R² is H, C₁₋₆alkyl, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆alkyl, C₁₋₆hetereoalkyl, or C₁₋₆alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring;

L is a bond, —CH₂(CO)—, —C₁₋₃alkylene-, —SO₂—, —C(O)—, —C(S)—, —C(NH)—, —C(O)NH—, —C(O)NHCH₂—, —C(O)N—, —C(O)OCH₂—, —C(O)O—, —C(O)C(O)—, —SO₂—NH—, or —CH₂C(O)—;

R³ is H, CN, oxo, C₁₋₆alkyl, C₅₋₁₄aryl, CH₂C₅₋₁₄aryl, CH₂C₃₋₇cycloalkyl, C₃₋₇cycloalkyl, C₃₋₇spirocycloalkyl, C₃₋₇cycloalkenyl, C₃₋₉heterocycle, or C₅₋₉heteroaryl, or R³ may join together with an R⁶ to form a fused 5-7 membered ring, and wherein each R³ group is optionally substituted by one to four substituents selected from halo, oxo, C₁₋₆alkyl, C₃₋₇cycloalkyl, C₁₋₃fluoroalkyl, —OC₁₋₆alkyl, —C(O)R⁴, —C(O)NR⁴, —C(O)NHR⁴, C₅₋₁₄aryl, C₁₋₆hetereoalkyl, —B(OH)₂, C₃₋₉heterocycle, C₅₋₉heteroaryl, —C(O)OC₁₋₆alkyl, or two substituents may bond together to form a fused, spiro, or bridged ring and that fused, spiro, or bridged ring may optionally be substituted with R⁴;

R⁴ is CN, halo, —OC₁₋₆alkyl, C₁₋₆alkyl, C₃₋₇cycloalkyl, C₃₋₉heterocycle, or C₅₋₁₄aryl;

each R⁵ is independently H, C₁₋₃alkyl, C₃₋₆cycloalkyl, CH₂F, CHF₂, or CF₃;

each R⁶ is independently H, or C₁₋₃alkyl, C₅₋₁₄aryl, C₃₋₉heterocycle, C₅₋₉heteroaryl, —C(O)NR⁴₇ or —C(O)NHR⁴, or both R⁶ may together comprise 2-4 carbon atoms and join together to form a bridged ring system;

and wherein each heterocycle, heteroaryl, heteroalkyl, and heteroalkylene comprises one to three heteroatoms selected from S, N, B, or O.

In another aspect the present invention discloses pharmaceutically acceptable salts of the compounds of Formula I.

In another aspect, the present invention discloses pharmaceutical compositions comprising a compound of Formula I or a pharmaceutically acceptable salt thereof.

In another aspect, the present invention discloses a method for treating a viral infection in a patient mediated at least in part by a virus in the retrovirus family of viruses, comprising administering to said patient a composition comprising a compound of Formula I, or a pharmaceutically acceptable salt thereof. In some embodiments, the viral infection is mediated by the HIV virus.

In another aspect, a particular embodiment of the present invention provides a method of treating a subject infected with HIV comprising administering to the subject a therapeutically effective amount of a compound of Formula I, or a pharmaceutically acceptable salt thereof.

In yet another aspect, a particular embodiment of the present invention provides a method of inhibiting progression of HIV infection in a subject at risk for infection with HIV comprising administering to the subject a therapeutically effective amount of a compound of Formula I, or a pharmaceutically acceptable salt thereof. Those and other embodiments are further described in the text that follows.

In accordance with another embodiment of the present invention, there is provided a method for preventing or treating a viral infection in a mammal mediated at least in part by a virus in the retrovirus family of viruses which method comprises administering to a mammal, that has been diagnosed with said viral infection or is at risk of developing said viral infection, a compound as defined in Formula I, wherein said virus is an HIV virus and further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

### DETAILED DESCRIPTION OF THE INVENTION

Preferably R¹ is C₁₋₆alkyl. Most preferably, R¹ is t-butyl.

Preferably X is O.

Preferably R² is optionally substituted phenyl. Most preferably, R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

Preferably R³ is C₁₋₆alkyl, phenyl, naphthyl, cyclopentyl, cyclohexyl, pyridyl, or tetrahydropyranyl, each of which is optionally substituted by 1-3 substituents selected from halogen, C₁₋₆alkyl, —OC₁₋₆alky, C₁₋₃fluoroalkyl, or phenyl.

Preferably each R⁵ is methyl.

Preferably each R⁶ is H.

Preferably the stereochemistry on the carbon to which XR¹ is bound is as depicted below.

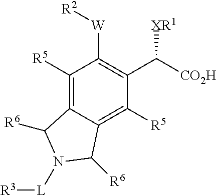

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

### EXAMPLES

The compounds of this invention may be made by a variety of methods, including well-known standard synthetic methods. Illustrative general synthetic methods are set out below and then specific compounds of the invention are prepared in the working examples.

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.

aq.=aqueous

μL=microliters

μM=micromolar

NMR=nuclear magnetic resonance

boc=tert-butoxycarbonyl

br=broad

Cbz=benzyloxycarbonyl

d=doublet

δ=chemical shift

oC=degrees celcius

DCM=dichloromethane

dd=doublet of doublets

DMEM=Dulbeco's Modified Eagle's Medium

DMF=N,N-dimethylformamide

DMSO=dimethylsulfoxide

EtOAc=ethyl acetate

g=gram

h or hr=hours

HCV=hepatitus C virus

HPLC=high performance liquid chromatography

Hz=hertz

IU=International Units

IC₅₀=inhibitory concentration at 50% inhibition

J=coupling constant (given in Hz unless otherwise indicated)

m=multiplet

M=molar

M+H⁺=parent mass spectrum peak plus H+

mg=milligram

min=minutes

mL=milliliter

mM=millimolar

mmol=millimole

MS=mass spectrum

nm=nanomolar

ppm=parts per million

q.s.=sufficient amount

s=singlet

RT=room temperature

sat.=saturated

t=triplet

TFA=trifluoroacetic acid

Z=benzyloxycarbonyl

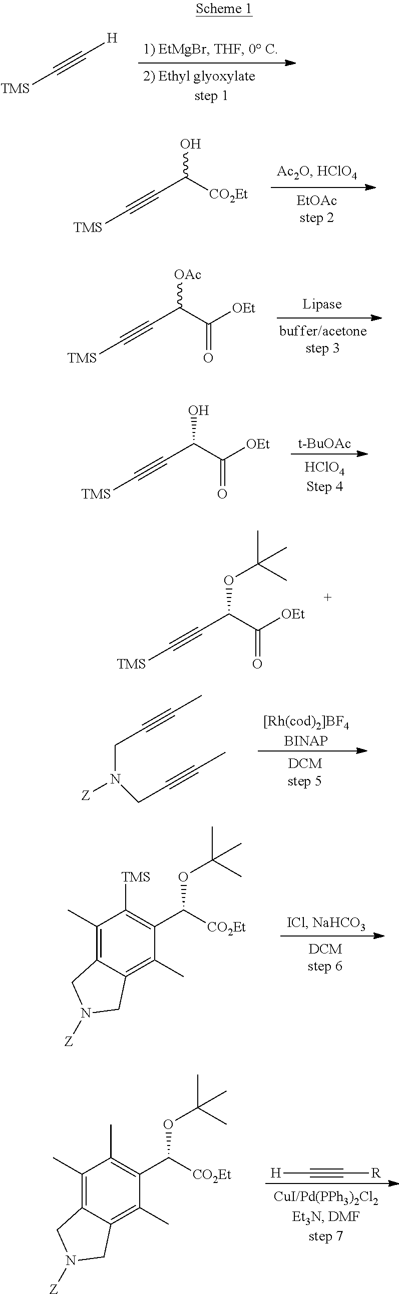

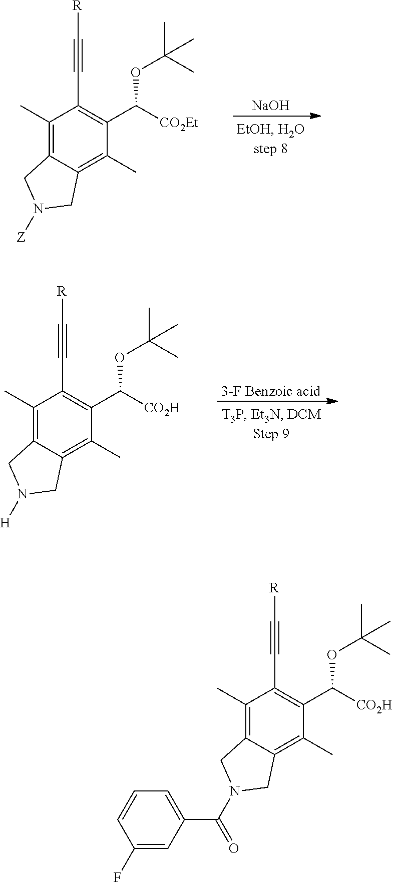

### Example 1

### (S)-2-(tert-butoxy)-2-(2-(3-fluorobenzoyl)-4,7-dimethyl-6-(phenylethynyl)isoindolin-5-yl)acetic acid

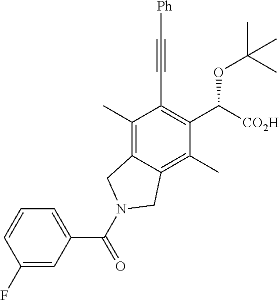 [SMILES: Cc1c(C#Cc2ccccc2)c([C@H](OC(C)(C)C)C(=O)O)c(C)c2c1CN(C(=O)c1cccc(F)c1)C2]

### Benzyl di(but-2-yn-1-yl)carbamate

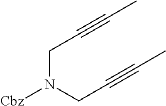 [SMILES: CC#CCN(CC#CC)C(=O)OCc1ccccc1]

To an ice-cooled solution of 1-bromobut-2-yne (581 g, 2.2 eq) in DMF (3.5 L) was added NaH (60%, 199 g, 2.5 eq) carefully and the mixture was stirred at 0° C. under N2 atmosphere for 15 min. Then a solution of benzyl carbamate (300 g, 1.985 mol, 1 eq) in DMF (500mL) was added dropwise at 0° C. for 1 h and the resulting mixture was allowed to warm to ambient temperature for 2h. After being quenched cautiously with H₂O, the reaction was extracted with ether (×2). The organic layer was washed with H₂O (×3), brine, dried over anhydrous Na₂SO₄, filtered, and concentrated. The residue was purified by column chromatography (silica gel, 0-5% EtOAc in hexane) to afford the title compound (398 g, 79%).¹H NMR (400 MHz, CDCl₃) δ 7.41-7.27 (m, 5H), 5.17 (s, 2H), 4.18 (s, 4H), 1.81 (t, J=2.3 Hz, 6H). LC-MS (ESI+): m/z (M+H)=256.3

### Step 1: Ethyl 2-hydroxy-4-(trimethylsilyl)but-3-ynoate

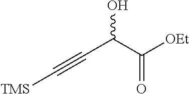 [SMILES: CCOC(=O)C(O)C#C[Si](C)(C)C]

To a solution of TMS-acetylene (250 g, 2.55 mol) in anhydrous THF (2.5 L) at 0° C. was added 3M EtMgBr/ether (933 mL, 2.80 mol) dropwise under an N₂ atmosphere while maintaining the inner temperature below 5° C. After stirring at 0° C. for 30 min, the suspension was added to an ice cold solution of 50% ethyl glyoxylate/toluene (624 g, 3.05 mol) in anhydrous THF (5 L) via cannula. After stirring at 0° C. for 1 h, the mixture was quenched with saturated aqueous NH₄Cl solution (3 L) and extracted with EtOAc (2×1 L). The combined EtOAc solutions were concentrated at reduced pressure. The residue was diluted with EtOAc (3 L). The solution was washed with water (2×1 L) and brine (2×1 L), dried over Na₂SO₄ and concentrated under reduced pressure. The crude material was purified by flash chromatography (silica gel, 0-10% EtOAc/petroleum ether) to give the title compound (285 g, 56%) as a yellow oil. ¹H NMR (400 MHz, CHLOROFORM-d) δ=4.83 (d, J=7.3 Hz, 1 H), 4.34 (qq, J=7.2, 10.8 Hz, 2H), 3.02 (d, J=7.3 Hz, 1H), 1.34 (t, J=7.2 Hz, 3H), 0.22-0.16 (m, 9H).

### Step 2: Ethyl 2-acetoxy-4-(trimethylsilyl)but-3-ynoate

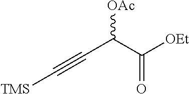 [SMILES: CCOC(=O)C(C#C[Si](C)(C)C)OC(C)=O]

To a 10 L flask was added EtOAc (7.5 L) followed by Ac₂O (400 mL). After stirring at RT for 30 minutes the mixture was cooled to 0° C. and treated with another portion of Ac₂O (2.1 L). After 1 hour at 0° C., the solution was allowed to warm to RT. To the solution was added ethyl 2-hydroxy-4-(trimethylsilyl)but-3-ynoate (520 g, 2.60 mol). After stirring at RT for 1 hour the solution was washed with 1N aqueous NaOH (3×, 20 L total). The solution was then washed with brine (5 L), dried over Na₂SO₄ and concentrated to dryness at reduced pressure. The crude product was purified by flash chromatography (silica gel, 0-5% EtOAc/petroleum ether) to give the title compound (590 g, 94%) as a yellow oil. ¹H NMR (400 MHz, CHLOROFORM-d) δ=5.69 (s, 1 H), 4.36-4.21 (m, 2H), 2.19 (s, 3H), 1.32 (t, J=7.2 Hz, 3H), 0.25-0.15 (m, 9H).

### Step 3: (S)-Ethyl 2-hydroxy-4-(trimethylsilyl)but-3-ynoate

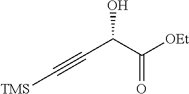 [SMILES: CCOC(=O)[C@@H](O)C#C[Si](C)(C)C]

To a solution of ethyl 2-acetoxy-4-(trimethylsilyl)but-3-ynoate (150 g, 0.620 mol) in acetone (1.88 L) and phosphate buffer solution (pH 7.2, 7.5 L) was added Amano Lipase PS (75 g). After stirring at 20° C. overnight, the reaction mixture was diluted with water (2.5 L) and extracted with EtOAc (3 L). The layers were separated and the organic layer was washed with brine (3×, 10 L total volume), dried over Na₂SO₄, filtered and concentrated under reduced pressure to give the crude product. This material was purified by flash chromatography (silica gel, 0-10% EtOAc/petroleum ether) to afford the title compound (55 g, 44%) as a yellow oil. ¹H NMR (400 MHz, CHLOROFORM-d) δ=4.83 (d, J=7.3 Hz, 1H), 4.34 (qq, J=7.2, 10.8 Hz, 2H), 3.02 (d, J=7.3 Hz, 1H), 1.34 (t, J=7.2 Hz, 3H), 0.22-0.16 (m, 9H).

### Step 4: (S)-Ethyl 2-(tert-butoxy)-4-(trimethylsilyl)but-3-ynoate

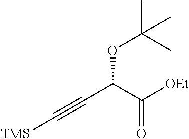 [SMILES: CCOC(=O)[C@H](C#C[Si](C)(C)C)OC(C)(C)C]

To a solution of (S)-ethyl 2-hydroxy-4-(trimethylsilyl)but-3-ynoate (100 g, 0.500 mol) in t-BuOAc (2.5 L) was added HClO₄ (41 mL, 0.500 mol) dropwise at RT. After stirring for 40 minutes, the mixture was quenched with NaHCO₃ powder, diluted with water (2 L) and extracted with EtOAc (2 L). The EtOAc solution was washed with brine, dried over Na₂SO₄, filtered and concentrated under reduced pressure to give the crude product. This material was was purified by flash chromatography (silica gel, 0-5% EtOAc/petroleum ether) to afford the title compound (103 g, 81%) as a yellow oil. ¹H NMR (400MHz, CHLOROFORM-d) δ=4.72 (s, 1H), 4.33-4.20 (m, 2H), 1.31 (t, J=7.2 Hz, 3H), 1.28 (s, 9H), 0.17 (s, 9H).

### Step 5: (S)-benzyl 5-(1-(tert-butoxy)-2-ethoxy-2-oxoethyl)-4,7-dimethyl-6-(trimethylsilyl)isoindoline-2-carboxylate

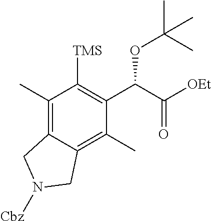 [SMILES: CCOC(=O)[C@@H](OC(C)(C)C)c1c(C)c2c(c(C)c1[Si](C)(C)C)CN(C(=O)OCc1ccccc1)C2]

A mixture of [Rh(cod)₂]BF₄ (5 g, 12.313 mmol, 6.3% eq) and (R)-BINAP (7.67 g, 6.3% eq) in DCM (50 mL) was stirred at ambient temperature under H₂ atmosphere for 6 h until the solution turned dark. This mixture was charged with N₂ and a solution of (S)-ethyl 2-tert-butoxy-4-(trimethylsilyl)but-3-ynoate (50 g, 195 mmol, 1 eq) in DCM (100 mL) was added. The mixture was heated to 40° C. A solution of benzyl di(but-2-yn-1-yl)carbamate (100 g, 390 mmol, 2 eq) in DCM (400mL) was added to the above mixture under N₂ atmosphere dropwise for 3 h and the resulting mixture was kept stirring at 40° C. for 0.5 h. After being concentrated to dryness, the residue was purified by column chromatography (silica gel, 0˜15% EtOAc in hexane) to afford the title compound (69 g, 69%). ¹H NMR (400 MHz, CDCl₃) δ 7.37 (ddd, J=14.0, 9.2, 6.6 Hz, 5H), 5.60 (s, 1H), 5.23 (s, 2H), 4.74-4.61 (m, 4H), 4.24 (dd, J=10.8, 7.1 Hz, 1H), 4.10 (s, 1H), 2.33 (d, J=12.9 Hz, 3H), 2.21 (d, J=10.6 Hz, 3H), 1.22 (td, J=7.1, 1.1 Hz, 3H), 1.16 (s, 9H), 0.48 (s, 9H). LC-MS (ESI+): m/z (M+H)=512.2

### Step 6: (S)-benzyl 5-(1-(tert-butoxy)-2-ethoxy-2-oxoethyl)-6-iodo-4,7-dimethylisoindoline-2-carboxylate

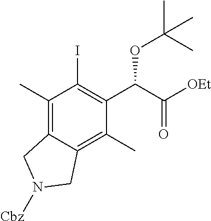 [SMILES: CCOC(=O)[C@@H](OC(C)(C)C)c1c(C)c2c(c(C)c1I)CN(C(=O)OCc1ccccc1)C2]

To an ice-cooled well-stirred suspension of (S)-benzyl 5-(1-tert-butoxy-2-ethoxy-2-oxoethyl)-4,7-dimethyl-6-(trimethylsilyl)isoindoline-2-carboxylate (90 g, 175.9 mmol, 1 eq) and sodium bicarbonate (296 g, 20 eq) in DCM (450mL) was added a solution of iodinemonochloride in DCM (1 M, 194 mL, 1.1 eq) at 0° C. under a N₂ atmosphere. The resulting mixture was stirred at 0° C. for 0.5 h. After being quenched with saturated sodium thiosulfate, the reaction mixture was extracted with EtOAc (1.5 L) and the organic layer was washed with brine, dried over anhydrous Na₂SO₄, filtered, and concentrated. The residue was purified by column chromatography (silica gel, 0-15% EtOAc in hexane) to afford the title compound (74 g, 74%). ¹H NMR (400 MHz, CDCl₃) δ 7.45-7.29 (m, 5H), 5.87 (s, 1H), 5.22 (s, 2H), 4.69 (dd, J=27.0, 15.3 Hz, 4H), 4.23-4.07 (m, 2H), 2.36 (d, J=12.2 Hz, 3H), 2.29 (d, J=10.7 Hz, 3H), 1.25-1.17 (m, 12H). LC-MS (ESI+): m/z (M+H)=566.1

### Step 7: Benzyl (S)-5-(1-(tert-butoxy)-2-ethoxy-2-oxoethyl)-4,7-dimethyl-6-(phenylethynyl)isoindoline-2-carboxylate

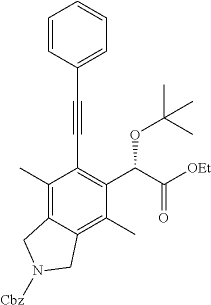 [SMILES: CCOC(=O)[C@@H](OC(C)(C)C)c1c(C)c2c(c(C)c1C#Cc1ccccc1)CN(C(=O)OCc1ccccc1)C2]

A mixture of benzyl (S)-5-(1-(tert-butoxy)-2-ethoxy-2-oxoethyl)-6-iodo-4,7-dimethylisoindoline-2-carboxylate (200 mg, 0.35 mmol), ethynylbenzene (145 mg, 1.41 mmol), Pd(dppf)Cl₂ (37.2 mg, 0.053 mmol) and Cul (20.2 mg, 0.106 mmol) in TEA (3 mL) was stirred at 60° C. under N₂ atmosphere overnight. The resulting mixture was filtered through a pad of Celite and the filtrate was partitioned between EtOAc and H₂O. The organic layer was washed with brine, dried over Na₂SO₄, filtered and concentrated under reduced pressure to give the crude product which was purified by silica gel chromatography (0-20% EtOAc in PE) to afford the title compound (35 mg, 18% yield) as a yellow oil. ¹H NMR (400 MHz, CDCl₃) δ 7.58-7.50 (m, 2H), 7.44-7.31 (m, 8H), 6.06 (s, 1H), 5.23 (s, 2H), 4.71 (d, J=15.0 Hz, 4H), 4.22-4.08 (m, 2H), 2.41 (d, J=11.6 Hz, 3H), 2.28 (d, J=10.4 Hz, 3H), 1.25 (d, J=1.2 Hz, 9H), 1.18 (td, J=7.1, 2.1 Hz, 3H). LC/MS (m/z) ES+=540.6 (M+1).

### Step 8: (S)-2-(tert-butoxy)-2-(4,7-dimethyl-6-(phenylethynyl)isoindolin-5-yl)acetic acid

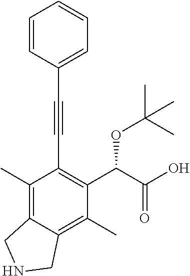 [SMILES: Cc1c(C#Cc2ccccc2)c([C@H](OC(C)(C)C)C(=O)O)c(C)c2c1CNC2]

A mixture of benzyl (S)-5-(1-(tert-butoxy)-2-ethoxy-2-oxoethyl)-4,7-dimethyl-6-(phenylethynyl)isoindoline-2-carboxylate (35 mg, 0.065 mmol) and NaOH (78 mg, 1.95 mmol) in EtOH (0.8 mL) and H₂O (0.4 mL) was stirred at 100° C. After 2 h, the reaction mixture was cooled to ambient temperature acidified with 1N HCl and extracted with EtOAc. The organic layer was washed with brine, dried over Na₂SO₄, filtered and concentrated under reduced pressure to give the title compound (25 mg, quant. yield) as a yellow oil which was used in the next step without further purification. LC/MS (m/z) ES+=378.4 (M+1).

### Step 9: (S)-2-(tert-butoxy)-2-(2-(3-fluorobenzoyl)-4,7-dimethyl-6-(phenylethynyl)isoindolin-5-yl)acetic acid

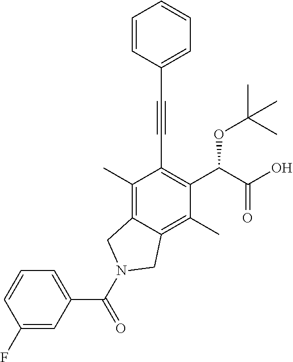 [SMILES: Cc1c(C#Cc2ccccc2)c([C@H](OC(C)(C)C)C(=O)O)c(C)c2c1CN(C(=O)c1cccc(F)c1)C2]

To a solution of (S)-2-(tert-butoxy)-2-(4,7-dimethyl-6-(phenylethynyl)isoindolin-5-yl)acetic acid (19 mg, 0.05 mmol) and 3-fluorobenzoic acid (14 mg, 0.1 mmol) in EtOAc (2 mL) was added propane phosphonic acid anhyrdide (80 mg, 0.126 mmol, 50% EtOAc solution) and Et₃N (15 mg, 0.15 mmol). After 1 h, the reaction mixture was quenched with sat. NaHCO₃ aq. solution and extracted with EtOAc. The organic layer was washed with brine, dried over Na₂SO₄, filtered and concentrated under reduced pressure to give the crude product which was purified by reverse phase HPLC (C18, 50-100% MeCN in H₂O with 0.1% formic acid) to afford the title compound (4.1 mg, 16% yield) as a yellow powder after lyophilization. ¹H NMR (400 MHz, DMSO) δ 12.29 (br, 1H), 7.49 (m, 9H), 5.87 (d, J=4.3 Hz, 1H), 4.83 (m, 4H), 2.28 (m, 6H), 1.18 (d, J=7.1 Hz, 9H). LC/MS (m/z) ES+=500.7 (M+1).

### Example 2

### (S)-2-(tert-butoxy)-2-(6-ethynyl-2-(3-fluorobenzoyl)-4,7-dimethylisoindolin-5-yl)acetic acid

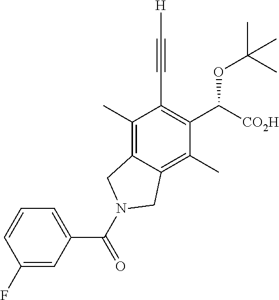 [SMILES: C#Cc1c(C)c2c(c(C)c1[C@H](OC(C)(C)C)C(=O)O)CN(C(=O)c1cccc(F)c1)C2]

The title compound was made in a similar manner as Example 1 except using TMS-acetylene in Step 7. ¹H NMR (400 MHz, DMSO) δ 12.47 (br, 1H), 7.55 (m, 1H), 7.46 (m, 2H), 7.35 (m, 1H), 5.81 (d, J=4.3 Hz, 1H), 4.76 (m, 5H), 2.21 (m, 6H), 1.15 (d, J=7.0 Hz, 9H). LC/MS (m/z) ES+=424.5 (M+1).

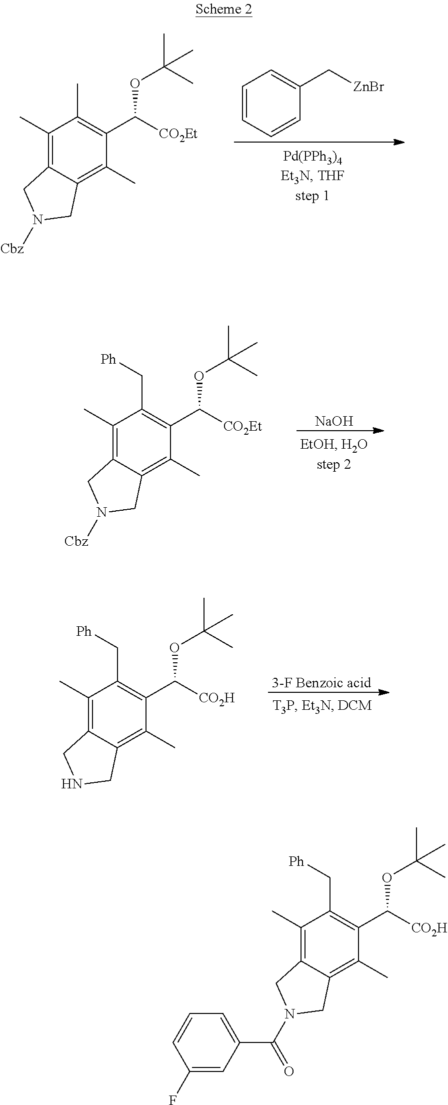

### Example 3

### (S)-2-(6-benzyl-2-(3-fluorobenzoyl)-4,7-dimethylisoindolin-5-yl)-2-(tert-butoxy)acetic acid

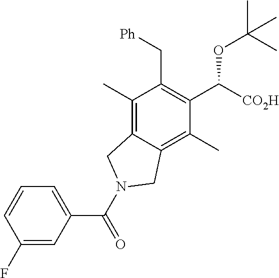 [SMILES: Cc1c2c(c(C)c([C@H](OC(C)(C)C)C(=O)O)c1Cc1ccccc1)CN(C(=O)c1cccc(F)c1)C2]

### Step 1. (S)-benzyl 5-benzyl-6-(1-(tert-butoxy)-2-ethoxy-2-oxoethyl)-4,7-dimethylisoindoline-2-carboxylate

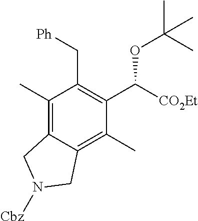 [SMILES: CCOC(=O)[C@@H](OC(C)(C)C)c1c(C)c2c(c(C)c1Cc1ccccc1)CN(C(=O)OCc1ccccc1)C2]

To an ice cold solution of benzyl (S)-5-(1-(tert-butwry)-2-ethoxy-2-oxoethyl)-6-iodo-4,7-dimethylisoindoline-2-carboxylate (135 mg, 0.24 mmol), Pd(PPh₃)₄ (56 mg, 0.0478 mmol) in THF (1 mL) was added benzylzinc(II) bromide (1 M, 0.48 mL, 0.48 mmol) and the reaction mixture was heated to 65° C. After 1 h, the reaction mixture was quenched with the addition of sat. NH₄Cl aq. solution and extracted with EtOAc. The organic layer was washed with brine, dried over Na₂SO₄, filtered and concentrated under reduced pressure. The residue was purified by silica gel chromatography (0-30% EtOAc in PE) to afford the title compound (122 mg, 97% yield) as a brown oil. LC/MS (m/z) ES+=530.7 (M+1).

### Step 2: (S)-2-(6-benzyl-4,7-dimethylisoindolin-5-yl)-2-(tert-butoxy)acetic acid

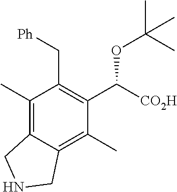 [SMILES: Cc1c2c(c(C)c([C@H](OC(C)(C)C)C(=O)O)c1Cc1ccccc1)CNC2]

A mixture of benzyl (S)-5-benzyl-6-(1-(tert-butwry)-2-ethoxy-2-oxoethyl)-4,7-dimethylisoindoline-2-carboxylate (115 mg, 0.16 mmol) and NaOH (193 mg, 4.8 mmol) in EtOH (2 mL) and H₂O (1 mL) was stirred at 100° C. under N₂ atmosphere. After 2 h, the reaction mixture was cooled to ambient temperature and the resulting mixture was acidified with 1N HCl and extracted with DCM/i-PrOH (85:15). The organic layer was washed with brine, dried over Na₂SO₄, filtered and concentrated under reduced pressure to give the title compound (80 mg, quant. yield) as a yellow oil which was used in the next step without further purification. LC/MS (m/z) ES+=368.5 (M+1).

### Step 3: (S)-2-(6-benzyl-2-(3-fluorobenzoyl)-4,7-dimethylisoindolin-5-yl)-2-(tert-butoxy)acetic acid

To a solution of (S)-2-(6-benzyl-4,7-dimethylisoindolin-5-yl)-2-(tert-butoxy)acetic acid (80 mg, 0.218 mmol) and 3-fluorobenzoic acid (61 mg, 0.435 mmol) in DCM (4 mL) and EtOAc (2 mL) was added propane phosphonic acid anhyrdide (346 mg, 0.544 mmol, 50% EtOAc solution) and Et₃N (66 mg, 0.653 mmol). After 1 h, the reaction mixture was quenched with sat. aq. NaHCO₃ and extracted with EtOAc. The organic layer was washed with brine, dried over Na₂SO₄, filtered and concentrated under reduced pressure. The residue was purified by reverse phase HPLC (C18, 50-100% MeCN in H₂O with 0.1% formic acid) to afford the title compound (21 mg, 20% yield) as a white powder. ¹H NMR (400 MHz, DMSO) δ 12.40 (br, 1H), 7.51 (m, 3H), 7.34 (m, 1H), 7.12 (m, 5H), 5.33 (s, 1H), 4.80 (dd, J=33.3, 22.2 Hz, 4H), 4.20 (m, 2H), 2.31 (d, J=66.6 Hz, 3H), 1.83 (d, J=64.5 Hz, 3H), 0.92 (s, 9H). LC/MS (m/z) ES+=490.6 (M+1).

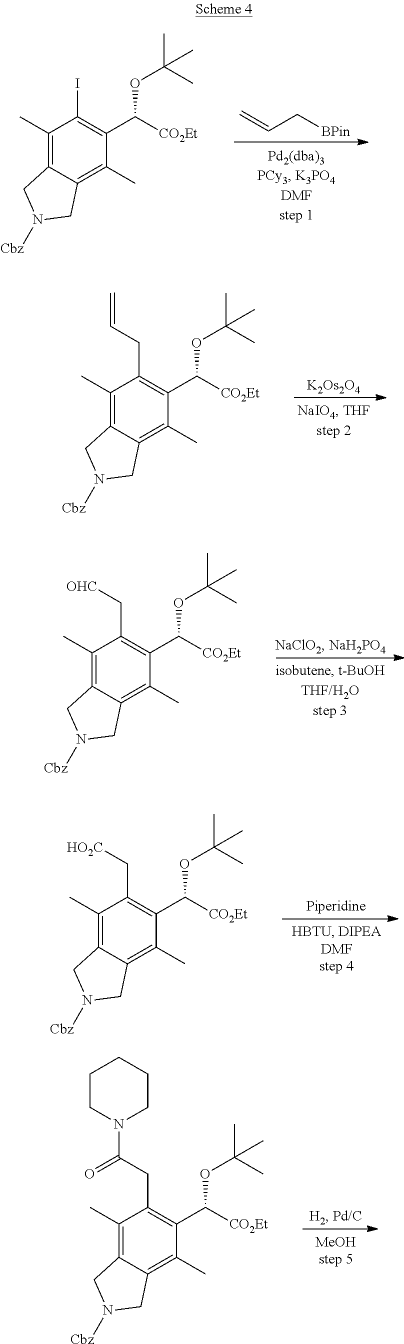

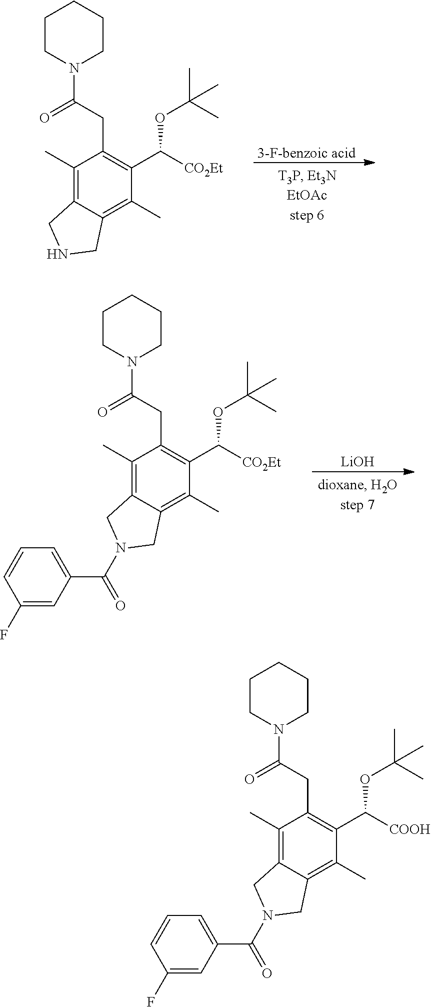 [SMILES: CCOC(=O)[C@@H](OC(C)(C)C)c1c(C)c2c(c(C)c1CC(=O)N1CCCCC1)CNC2 ; "3-F-benzoic acid" ; CCOC(=O)[C@@H](OC(C)(C)C)c1c(C)c2c(c(C)c1CC(=O)N1CCCCC1)CN(C(=O)c1cccc(F)c1)C2 ; "LiOH" ; Cc1c2c(c(C)c([C@H](OC(C)(C)C)C(=O)O)c1CC(=O)N1CCCCC1)CN(C(=O)c1cccc(F)c1)C2]

### Example 4

### (S)-2-(tert-butoxy)-2-(2-(3-fluorobenzoyl)-4,7-dimethyl-6-(2-oxo-2-(piperidin-1-yl)ethyl)isoindolin-5-yl)acetic acid

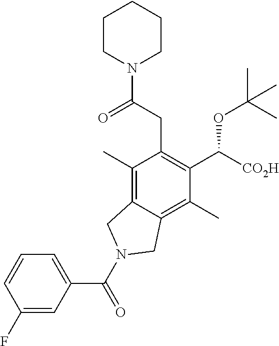 [SMILES: Cc1c2c(c(C)c([C@H](OC(C)(C)C)C(=O)O)c1CC(=O)N1CCCCC1)CN(C(=O)c1cccc(F)c1)C2]

### Step 1: (S)-benzyl 5-allyl-6-(1-(tert-butoxy)-2-ethoxy-2-oxoethyl)-4,7-dimethylisoindoline-2-carboxylate

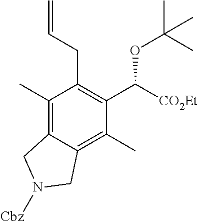 [SMILES: C=CCc1c(C)c2c(c(C)c1[C@H](OC(C)(C)C)C(=O)OCC)CN(C(=O)OCc1ccccc1)C2]

A mixture of benzyl (S)-5-(1-(tert-butoxy)-2-ethoxy-2-oxoethyl)-6-iodo-4,7-dimethylisoindoline-2-carboxylate (1 g, 1.77 mmol), 2-allyl-4,4,5,5-tetramethyl-1,3,2-dioxaborolane (386 mg, 2.3 mmol), Pd₂(dba)₃ (324 mg, 0.35 mmol), PCy₃ (99 mg, 0.35 mmol) and K₃PO₄ (1.1 g, 5.3 mmol) in DMF was stirred at 80° C. After 18 h, the reaction mixture was cooled to ambient temperature and partitioned between EtOAc and H₂O. The organic layer was washed with brine, dried over Na₂SO₄, filtered and concentrated under reduced pressure to give the crude product which was purified by ISCO (silica gel, 0-20% EtOAc in PE) to afford the title compound (680 mg, 80% yield) as a colorless oil. LC/MS (m/z) ES+=480.6 (M+1).

### Step 2: (S)-benzyl 5-(1-(tert-butoxy)-2-ethoxy-2-oxoethyl)-4,7-dimethyl-6-(2-oxoethyl)isoindoline-2-carboxylate

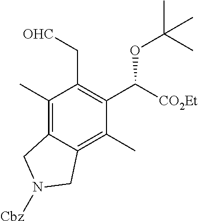 [SMILES: CCOC(=O)[C@@H](OC(C)(C)C)c1c(C)c2c(c(C)c1CC=O)CN(C(=O)OCc1ccccc1)C2]

To a solution of benzyl (S)-5-allyl-6-(1-(tert-butoxy)-2-ethoxy-2-oxoethyl)-4,7-dimethylisoindoline-2-carboxylate (680 mg, 1.4 mmol) in THF (8 mL) and H₂O (4 mL) was added NalO₄ (1.2 g, 5.7 mmol) and K₂Os₂O₄ (80 mg, 0.21 mmol). After 18 h, the reaction mixture was partitioned between EtOAc and H₂O. The organic layer was washed with sat. aq. Na₂S₂O₃ and brine, dried over Na₂SO₄, filtered and concentrated under reduced pressure. The residue was purified on silica gel (silica gel, 0-30% EtOAc in PE) to afford the title compound (300 mg, 44% yield) as a colorless oil. ¹H NMR (400 MHz, CDCl₃) δ 9.72 (s, 1H), 7.47-7.30 (m, 5H), 5.44 (d, J=5.9 Hz, 1H), 5.23 (s, 2H), 4.81-4.67 (m, 4H), 4.43 (dd, J=18.0, 3.0 Hz, 1H), 4.22-4.13 (m, 1H), 4.07-3.97 (m, 1H), 3.87 (dd, J=18.1, 4.1 Hz, 1H), 2.36 (d, J=11.7 Hz, 3H), 2.04 (d, J=12.5 Hz, 3H), 1.23-1.15 (m, 12H). LC/MS (m/z) ES+=482.4 (M+1).

### Step 3: (S)-2-(2-((benzyloxy)carbonyl)-6-(1-(tert-butoxy)-2-ethoxy-2-oxoethyl)-4,7-dimethylisoindolin-5-yl)acetic acid

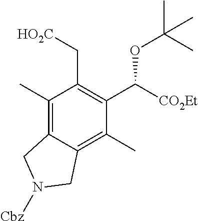 [SMILES: CCOC(=O)[C@@H](OC(C)(C)C)c1c(C)c2c(c(C)c1CC(=O)O)CN(C(=O)OCc1ccccc1)C2]

A mixture of benzyl (S)-5-(1-(tert-butoxy)-2-ethoxy-2-oxoethyl)-4,7-dimethyl-6-(2-oxoethyl)isoindoline-2-carboxylate (300 mg, 0.62 mmol), NaClO₂ (449 mg, 4.96 mmol), NaH₂PO₄ (446 mg, 3.7 mmol) in isobutene (4 mL), THF (2 mL), t-BuOH (2 mL) and H₂O (2 mL) was stirred at ambient temperature. After 2 h, the reaction mixture was partitioned between EtOAc and H₂O. The organic layer was washed with sat. aq. Na₂S₂O₃ and brine, dried over Na₂SO₄, filtered and concentrated under reduced pressure. The residue was purified by silica gel chromatography (silica gel, 0-10% MeOH in DCM) to afford the title compound (120 mg, 39% yield) as a colorless oil. LC/MS (m/z) ES+=498.4 (M+1).

### Step 4: (S)-benzyl 5-(1-(tert-butoxy)-2-ethoxy-2-oxoethyl)-4,7-dimethyl-6-(2-oxo-2-(piperidin-1-yl)ethyl)isoindoline-2-carboxylate

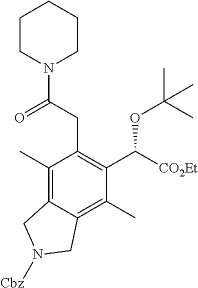 [SMILES: CCOC(=O)[C@@H](OC(C)(C)C)c1c(C)c2c(c(C)c1CC(=O)N1CCCCC1)CN(C(=O)OCc1ccccc1)C2]

A mixture of (S)-2-(2-((benzyloxy)carbonyl)-6-(1-(tert-butoxy)-2-ethoxy-2-oxoethyl)-4,7-dimethyl isoindolin-5-yl)acetic acid (50 mg, 0.1 mmol), piperidine (10 mg, 0.12 mmol), HBTU (59 mg, 0.15 mmol) and DIPEA (0.5 mL) in DMF (2 mL) was stirred at ambient temperature. After 1 h, the resulting mixture was quenched with sat. aq. NaHCO₃ and extracted with EtOAc. The organic layer was washed with brine, dried over Na₂SO₄, filtered and concentrated under reduced pressure. The residue was purified by silica gel chromatography (silica gel, 0-30% EtOAc in PE) to afford the title compound (40 mg, 70% yield) as a yellow oil. LC/MS (m/z) ES+=565.7 (M+1).

### Step 5: (S)-2-(tert-butoxy)-2-(2-(3-fluorobenzoyl)-4,7-dimethyl-6-(2-oxo-2-(piperidin-1-yl)ethyl)isoindolin-5-yl)acetic acid

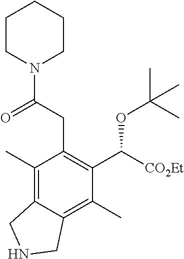 [SMILES: CCOC(=O)[C@@H](OC(C)(C)C)c1c(C)c2c(c(C)c1CC(=O)N1CCCCC1)CNC2]

A mixture of benzyl (S)-5-(1-(tert-butoxy)-2-ethoxy-2-oxoethyl)-4,7-dimethyl-6-(2-oxo-2-(piperidin-1-yl)ethyl)isoindoline-2-carboxylate (43 mg, 0.08 mmol) and 10% Pd/C (43 mg) in MeOH (4 mL) was hydrogenated under an atmosphere of H₂ (1 atm). After 1 h, the reaction mixture was filtered through a pad of Celite and the residue was concentrated under reduced pressure to give the crude title product (35 mg, quant. yield) as a brown oil which was used in the next step without further purification. LC/MS (m/z) ES+=431.4 (M+1).

### Step 6: Ethyl (S)-2-(tert-butoxy)-2-(2-(3-fluorobenzoyI)-4,7-dimethyl-6-(2-oxo-2-(piperidin-1-yl)ethyl)isoindolin-5-yl)acetate

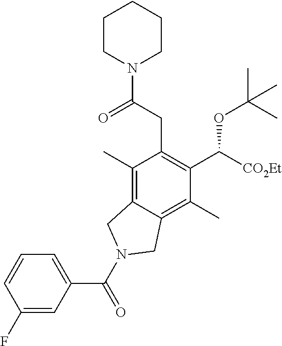 [SMILES: CCOC(=O)[C@@H](OC(C)(C)C)c1c(C)c2c(c(C)c1CC(=O)N1CCCCC1)CN(C(=O)c1cccc(F)c1)C2]

To a solution of (S)-2-(tert-butoxy)-2-(2-(3-fluorobenzoyl)-4,7-dimethyl-6-(2-oxo-2-(piperidin-1-yl)ethyl)isoindolin-5-yl)acetic acid (35 mg, 0.08 mmol, crude product from the previous step) and 3-fluorobenzoic acid (25 mg, 0.16 mmol) in EtOAc (3 mL) was added propane phosphonic acid anhyrdide (141 mg, 0.2 mmol, 50% EtOAc solution) and Et₃N (0.1, 0.49 mmol). After 1 h, the reaction mixture was quenched with sat. aq. NaHCO₃ and extracted with EtOAc. The organic layer was washed with half saturated aq. citric acid and brine, dried over Na₂SO₄, filtered and concentrated under reduced pressure. The residue was purified by silica gel chromatography (0-30% EtOAc in PE) to afford the title compound (22 mg, 50% yield) as a yellow oil. LC/MS (m/z) ES+=553.8 (M+1).

### Step 7: (S)-2-(tert-butoxy)-2-(2-(3-fluorobenzoyl)-4,7-dimethyl-6-(2-oxo-2-(piperidin-1-yl)ethyl)isoindolin-5-yl)acetic acid

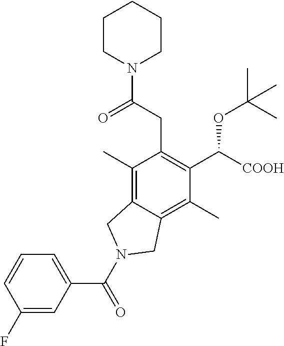 [SMILES: Cc1c2c(c(C)c([C@H](OC(C)(C)C)C(=O)O)c1CC(=O)N1CCCCC1)CN(C(=O)c1cccc(F)c1)C2]

A mixture of ethyl (S)-2-(tert-butoxy)-2-(2-(3-fluorobenzoyl)-4,7-dimethyl-6-(2-oxo-2-(piperidin-1-yl)ethyl)isoindolin-5-yl)acetate (22 mg, 0.04 mmol) in LiOH (0.24 mL, 2.5 N) and dioxane (1.5 mL) was stirred at 80° C. After 18 h, the reaction mixture was cooled to ambient temperature, diluted with EtOAc and washed with 1N HCl. The organic layer was washed with brine, dried over Na₂SO₄, filtered and concentrated under reduced pressure. The residue was purified by reverse phase HPLC (C18, 40-100% MeCN in H₂O with 0.1% formic acid) to afford the title compound (9 mg, 44% yield) as a white powder. ¹H NMR (400 MHz, DMSO) δ 12.61 (br, 1H), 7.58-7.31 (m, 4H), 5.25 (d, J=18.1 Hz, 1H), 4.78 (dd, J=35.2, 11.6 Hz, 4H), 3.69-3.37 (m, 6H), 2.27 (d, J=66.5 Hz, 3H), 1.92 (d, J=64.8 Hz, 3H), 1.66-1.37 (m, 6H), 1.06 (d, J=6.9 Hz, 9H). LC/MS (m/z) ES+=525.6 (M+1).

Examples 5-7 were prepared in a manner similar to the procedures described for Example 4.

### Example 5

### (S)-2-(tert-butoxy)-2-(6-(2-(cyclohexylamino)-2-oxoethyl)-2-(3-fluorobenzoyl)-4,7-dimethylisoindolin-5-yl)acetic acid

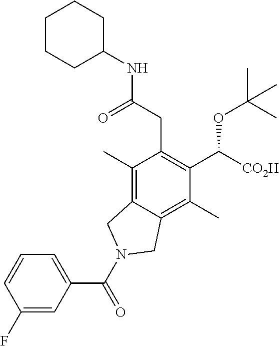 [SMILES: Cc1c2c(c(C)c([C@H](OC(C)(C)C)C(=O)O)c1CC(=O)NC1CCCCC1)CN(C(=O)c1cccc(F)c1)C2]

¹H NMR (400 MHz, DMSO) δ 12.29 (br, 1H), 7.54 (dd, J=13.7, 8.0 Hz, 1H), 7.45 (m, 2H), 7.35 (t, J=8.3 Hz, 1 H), 7.20 (s, 1 H), 6.65 (s, 1H), 5.30 (m, 2H), 4.76 (d, J=34.8 Hz, 4H), 3.97 (m, 1H), 2.26 (d, J=67.1 Hz, 3H), 2.00 (m, 5H), 1.49 (m, 7H), 1.10 (d, J=8.6 Hz, 9H), 0.86 (m, 1H). LC/MS (m/z) ES+=539.3 (M+1).

### Example 6

### (S)-2-(tert-butoxy)-2-(2-(3-fluorobenzoyl)-4,7-dimethyl-6-(2-oxo-2-(phenylamino)ethyl)isoindolin-5-yl)acetic acid

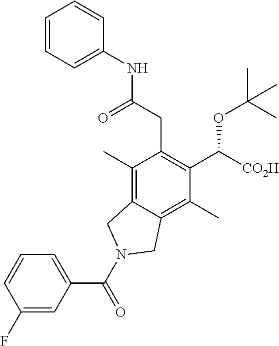 [SMILES: Cc1c2c(c(C)c([C@H](OC(C)(C)C)C(=O)O)c1CC(=O)Nc1ccccc1)CN(C(=O)c1cccc(F)c1)C2]

¹H NMR (400 MHz, DMSO) δ 12.41 (br, 1H), 9.79 (br, 1H), 7.62-7.50 (m, 3H), 7.50-7.42 (m, 2H), 7.35 (t, J=8.1 Hz, 1H), 7.26 (dd, J=13.5, 7.5 Hz, 2H), 6.98 (dd, J=12.5, 6.7 Hz, 1H), 5.37 (d, J=16.7 Hz, 1H), 4.92-4.68 (m, 4H), 4.16-3.80 (m, 2H), 2.37-1.93 (m, 6H), 1.05 (d, J=8.0 Hz, 9H). LC/MS (m/z) ES+=533.1 (M+1).

### Example 7

### (S)-2-(tert-butoxy)-2-(2-(3-fluorobenzoyl)-4,7-dimethyl-6-(2-(neopentylamino)-2-oxoethyl)isoindolin-5-yl)acetic acid

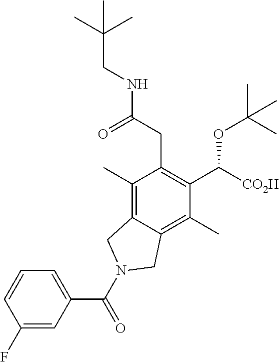 [SMILES: Cc1c2c(c(C)c([C@H](OC(C)(C)C)C(=O)O)c1CC(=O)NCC(C)(C)C)CN(C(=O)c1cccc(F)c1)C2]

¹H NMR (400 MHz, DMSO) δ 12.45 (br, 1H), 8.20 (br, 1H), 7.57-7.51 (m, 1H), 7.49-7.41 (m, 2H), 7.35 (t, J=8.0 Hz, 1H), 5.36 (d, J=13.8 Hz, 1 H), 4.88-4.67 (m, 4H), 4.01-3.72 (m, 2H), 2.88 (d, 2H), 2.34-1.92 (m, 6H), 1.10 (d, J=8.7 Hz, 9H), 0.82 (d, J=9.2 Hz, 9H). LC/MS (m/z) ES+=527.2 (M+1).

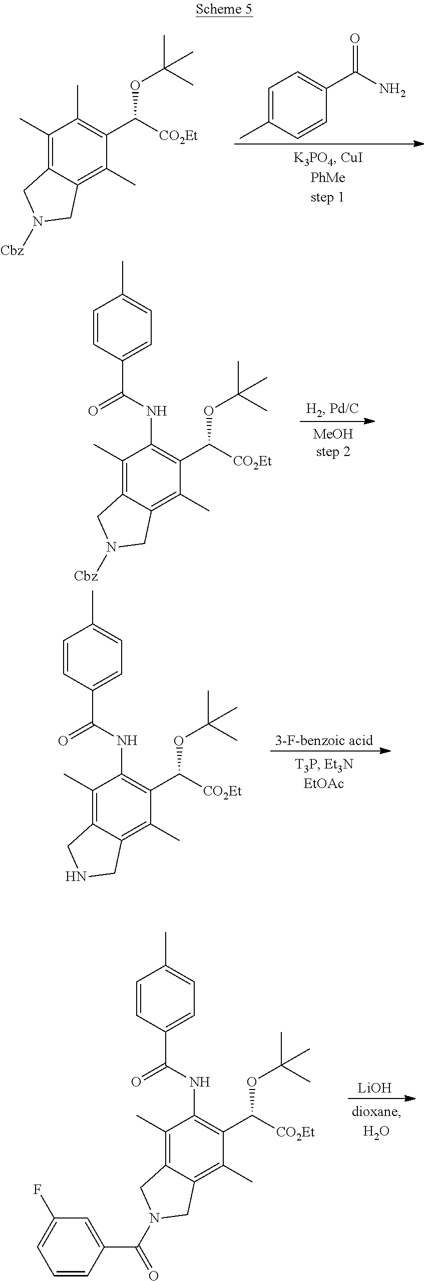

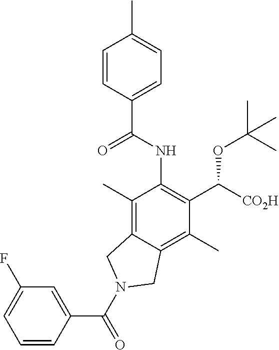 [SMILES: Cc1ccc(C(=O)Nc2c(C)c3c(c(C)c2[C@H](OC(C)(C)C)C(=O)O)CN(C(=O)c2cccc(F)c2)C3)cc1]

### Example 8

### (S)-2-(tert-butoxy)-2-(2-(3-fluorobenzoyl)-4,7-dimethyl-6-(4-methylbenzamido)isoindolin-5-yl)acetic acid

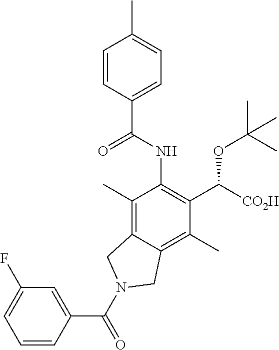 [SMILES: Cc1ccc(C(=O)Nc2c(C)c3c(c(C)c2[C@H](OC(C)(C)C)C(=O)O)CN(C(=O)c2cccc(F)c2)C3)cc1]

### Step 1: (S)-benzyl 5-(1-(tert-butoxy)-2-ethoxy-2-oxoethyl)-4,7-dimethyl-6-(4-methylbenzamido)isoindoline-2-carboxylate

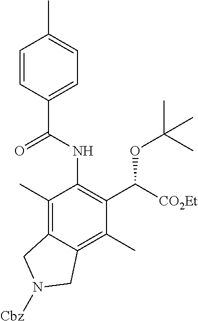 [SMILES: CCOC(=O)[C@@H](OC(C)(C)C)c1c(C)c2c(c(C)c1NC(=O)c1ccc(C)cc1)CN(C(=O)OCc1ccccc1)C2]

A mixture of benzyl (S)-5-(1-(tert-butoxy)-2-ethoxy-2-oxoethyl)-6-iodo-4,7-dimethylisoindoline-2-carboxylate (300 mg, 0.53 mmol), 4-methylbenzamide (143 mg, 1.06 mmol), N¹,N²-dimethylethane-1,2-diamine (18 mg, 0.21 mmol), CuI (20 mg, 0.11 mmol) and K₃PO₄ (338 mg, 1.59 mmol) in toluene (6 mL) was stirred at 110° C. After 24 h, the reaction mixture was cooled to ambient temperature and partitioned between EtOAc and H₂O. The layers were separated and the organic layer was washed with brine, dried over Na₂SO₄, filtered and concentrated under reduced pressure. The residue was purified by silica gel chromatography (0-30% EtOAc in PE) to afford the title compound (80 mg, 48% yield) as a light yellow oil. ¹H NMR (400 MHz, CDCl₃) δ 9.46 (d, J=7.6 Hz, 1H), 7.94 (d, J=7.9 Hz, 2H), 7.46-7.28 (m, 7H), 5.41 (d, J=4.0 Hz, 1 H), 5.24 (s, 2H), 4.82-4.67 (m, 4H), 4.02-3.91 (m, 2H), 2.42 (s, 3H), 2.34 (d, J=11.6 Hz, 3H), 2.12 (d, J=11.2 Hz, 3H), 1.27-1.18 (m, 12H). LC/MS (m/z) ES+=573.7 (M+1).

### Step 2: (S)-ethyl 2-(tert-butoxy)-2-(4, 7-dimethyl-6-(4-methylbenzamido)isoindolin-5-yl)acetate

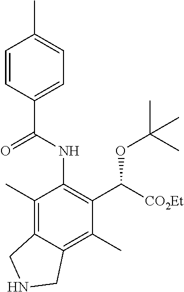 [SMILES: CCOC(=O)[C@@H](OC(C)(C)C)c1c(C)c2c(c(C)c1NC(=O)c1ccc(C)cc1)CNC2]

A mixture of benzyl (S)-5-(1-(tert-butoxy)-2-ethoxy-2-oxoethyl)-4,7-dimethyl-6-(4-methyl benzamido)isoindoline-2-carboxylate (80 mg, 0.14 mmol) and 10% Pd/C (80 mg) in MeOH (6 mL) was hydrogenated under a H2 atmosphere (1 atm). After 1 h, the resulting mixture was filtered through a pad of Celite and the residue was concentrated under reduced pressure to give the crude title product (50 mg, 81% yield) as a brown oil which was used in the next step without further purification. LC/MS (m/z) ES+=439.5 (M+1).

### Step 3: Ethyl (S)-2-(tert-butoxy)-2-(2-(3-fluorobenzoyl)-4,7-dimethyl-6-(4-methylbenzamido)isoindolin-5-yl)acetate

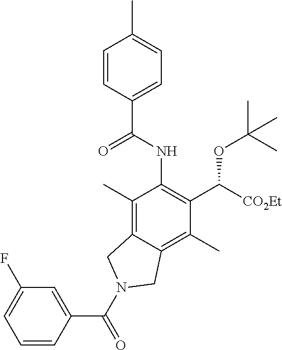 [SMILES: CCOC(=O)[C@@H](OC(C)(C)C)c1c(C)c2c(c(C)c1NC(=O)c1ccc(C)cc1)CN(C(=O)c1cccc(F)c1)C2]

To a solution of ethyl (S)-2-(tert-butoxy)-2-(4,7-dimethyl-6-(4-methylbenzamido)isoindolin-5-yl)acetate (50 mg, 0.11 mmol, crude product from the previous step) and 3-fluorobenzoic acid (32 mg, 0.23 mmol) in EtOAc (3 mL) was added propane phosphonic acid anhyrdide (181 mg, 0.29 mmol, 50% EtOAc solution) and Et₃N (87 mg, 0.86 mmol). After 2 h, the reaction mixture was quenched with sat. aq. NaHCO₃ and extracted with EtOAc. The organic layer was washed with half saturated aq. citric acid and brine, dried over Na₂SO₄, filtered and concentrated under reduced pressure. The residue was purified by silica gel chromatography (0-40% EtOAc in PE) to afford the title compound (35 mg, 55% yield) as a yellow oil. LC/MS (m/z) ES+=561.6 (M+1).

### Step 4: (S)-2-(tert-butoxy)-2-(2-(3-fluorobenzoyl)-4,7-dimethyl-6-(4-methylbenzamido)isoindolin-5-yl)acetic acid

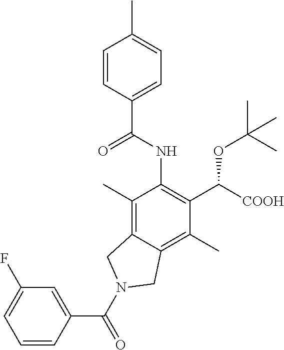 [SMILES: Cc1ccc(C(=O)Nc2c(C)c3c(c(C)c2[C@H](OC(C)(C)C)C(=O)O)CN(C(=O)c2cccc(F)c2)C3)cc1]

A mixture of ethyl (S)-2-(tert-butoxy)-2-(2-(3-fluorobenzoyl)-4,7-dimethyl-6-(4-methylbenzamido) isoindolin-5-yl)acetate (35 mg, 0.06 mmol) and LiOH (23 mg, 0.94 mmol) in dioxane (2 mL) and H₂O (0.5 mL) was stirred at 80° C. After 18 h, the resulting mixture was diluted with EtOAc and acidified with 1N HCl. The organic layer was washed with brine, dried over Na₂SO₄, filtered and concentrated under reduced pressure. The residue was purified by reverse phase HPLC (C18, 0-100% MeCN in H₂O with 0.1% formic acid) to afford the title compound (13 mg, 39% yield) as a white powder. ¹H NMR (400 MHz, DMSO) δ 12.62 (br, 1H), 9.73 (br, 1H), 7.90 (t, J=7.5 Hz, 2H), 7.53 (m, 3H), 7.34 (m, 3H), 5.35 (d, J=11.7 Hz, 1 H), 4.82 (dd, J=31.7, 5.6 Hz, 4H), 2.33 (m, 6H), 1.97 (d, J=62.2 Hz, 3H), 1.09 (d, J=5.6 Hz, 9H). LC/MS (m/z) ES+=533.6 (M+1).

Examples 9-11 were prepared in a manner similar to the procedures described for Example 8.

### Example 9

### (S)-2-(tert-butoxy)-2-(6-(2-cyclohexylacetamido)-2-(3-fluorobenzoyl)-4,7-dimethylisoindolin-5-yl)acetic acid

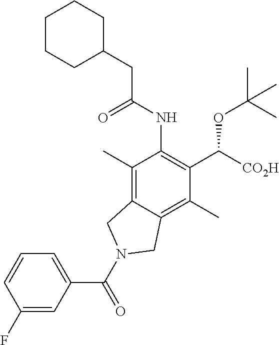 [SMILES: Cc1c2c(c(C)c([C@H](OC(C)(C)C)C(=O)O)c1NC(=O)CC1CCCCC1)CN(C(=O)c1cccc(F)c1)C2]

¹H NMR (400 MHz, DMSO) δ 12.32 (br, 1H), 9.12 (br, 1H), 7.54 (dd, J=13.8, 7.4 Hz, 1H), 7.46 (d, J=7.8 Hz, 2H), 7.35 (t, J=8.1 Hz, 1H), 5.31 (s, 1H), 4.78 (d, J=32.2 Hz, 4H), 2.23 (m, 5H), 1.94 (d, J=63.4 Hz, 3H), 1.70 (m, 6H), 1.11 (m, 14H). LC/MS (m/z) ES+=539.8 (M+1).

### Example 10

### (S)-2-(6-benzamido-2-(3-fluorobenzoyl)-4,7-dimethylisoindolin-5-yl)-2-(tert-butoxy)acetic acid

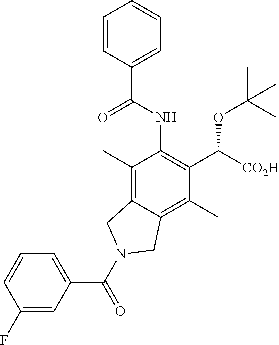 [SMILES: Cc1c2c(c(C)c([C@H](OC(C)(C)C)C(=O)O)c1NC(=O)c1ccccc1)CN(C(=O)c1cccc(F)c1)C2]

¹H NMR (400 MHz, DMSO) δ 12.66 (br, 1H), 8.13 (m, 3H), 7.53 (m, J=26.7, 13.8, 7.6 Hz, 6H), 7.35 (t, J=8.5 Hz, 1H), 5.23 (s, 1 H), 4.82 (m, 4H), 2.28 (d, J=65.3 Hz, 3H), 1.98 (d, J=62.2 Hz, 3H), 1.00 (s, 9H). LC/MS (m/z) ES+=519.6 (M+1).

### Example 11

### (S)-2-(tert-butoxy)-2-(2-(3-fluorobenzoyl)-4,7-dimethyl-6-(piperidine-1-carboxamido)isoindolin-5-yl)acetic acid

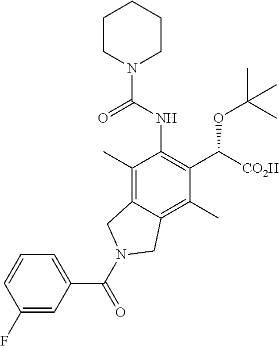 [SMILES: Cc1c2c(c(C)c([C@H](OC(C)(C)C)C(=O)O)c1NC(=O)N1CCCCC1)CN(C(=O)c1cccc(F)c1)C2]

¹H NMR (400 MHz, DMSO) δ 12.49 (br, 1H), 8.36 (br, 1H), 7.54 (m, 1H), 7.46 (d, J=7.9 Hz, 2H), 7.35 (dd, J=9.7, 7.3 Hz, 1H), 5.30 (m, 1H), 4.77 (m, 4H), 3.42 (m, 4H), 2.24 (d, J=65.7 Hz, 3H), 1.92 (d, J=67.1 Hz, 3H), 1.57 (m, 6H), 1.12 (d, J=6.3 Hz, 9H). LC/MS (m/z) ES+=526.6 (M+1).

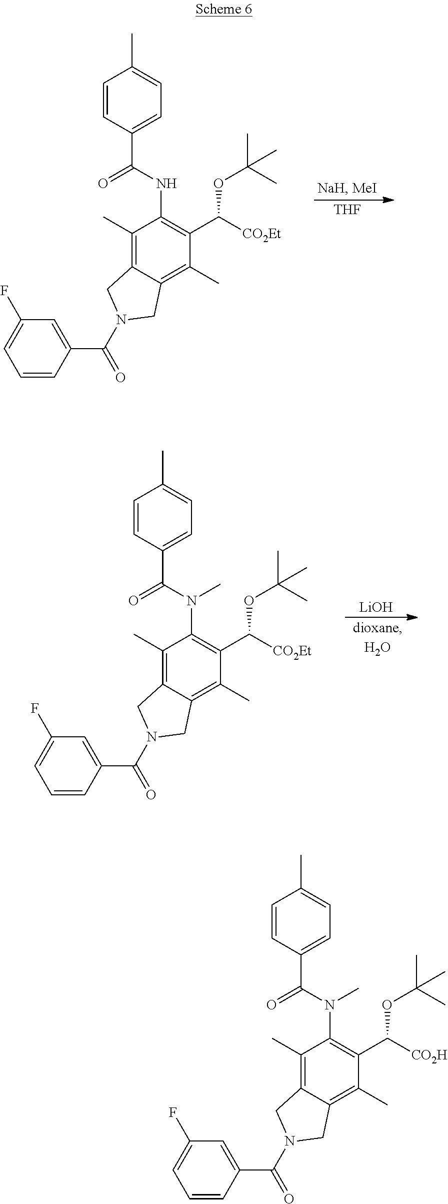

### Example 12

### (S)-2-(tert-butoxy)-2-(6-(N,4-dimethylbenzamido)-2-(3-fluorobenzoyl)-4,7-dimethylisoindolin-5-yl)acetic acid

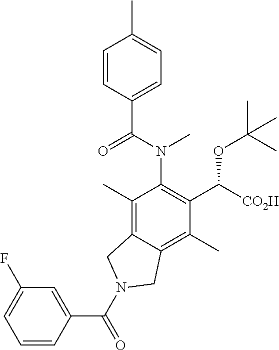 [SMILES: Cc1ccc(C(=O)N(C)c2c(C)c3c(c(C)c2[C@H](OC(C)(C)C)C(=O)O)CN(C(=O)c2cccc(F)c2)C3)cc1]

### Step 1: (S)-ethyl 2-(tert-butoxy)-2-(6-(N,4-dimethylbenzamido)-2-(3-fluorobenzoyl)-4,7-dimethylisoindolin-5-yl)acetate

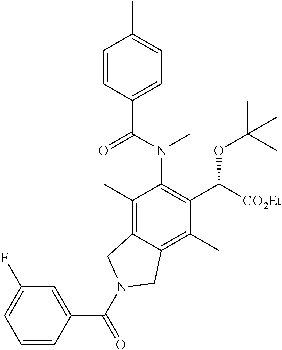 [SMILES: CCOC(=O)[C@@H](OC(C)(C)C)c1c(C)c2c(c(C)c1N(C)C(=O)c1ccc(C)cc1)CN(C(=O)c1cccc(F)c1)C2]

An ice cold solution of ethyl (S)-2-(tert-butoxy)-2-(2-(3-fluorobenzoyl)-4,7-dimethyl-6-(4-methyl benzamido)isoindolin-5-yl)acetate (50 mg, 0.09 mmol) in THF (2 mL) was treated with NaH (60%, 18 mg, 0.45 mmol). After 30 min, Mel (126 mg, 0.89 mmol) was added and the reaction mixture was warmed to ambient temperature. After 1 h, the reaction mixture was quenched with sat. aq. NH₄Cl and extracted with EtOAc. The organic layer was washed with brine, dried over Na₂SO₄, filtered and concentrated under reduced pressure. The residue was purified by silica gel chromatography (0-30% EtOAc in PE) to afford the title compound (30 mg, 59% yield) as a yellow oil. LC/MS (m/z) ES+=575.8 (M+1).

### Step 2: (S)-2-(tert-butoxy)-2-(6-(N, 4-dimethylbenzamido)-2-(3-fluorobenzoyl)-4,7-dimethylisoindolin-5-yl)acetic acid

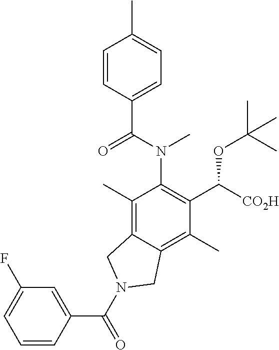 [SMILES: Cc1ccc(C(=O)N(C)c2c(C)c3c(c(C)c2[C@H](OC(C)(C)C)C(=O)O)CN(C(=O)c2cccc(F)c2)C3)cc1]

A mixture of ethyl (S)-2-(tert-butoxy)-2-(6-(N,4-dimethylbenzamido)-2-(3-fluorobenzoyl)-4,7-dimethylisoindolin-5-yl)acetate (60 mg, 0.10 mmol) and LiOH (38 mg, 1.57 mmol) in dioxane (3 mL) and H₂O (0.7 mL) was stirred at 80° C. After 18 h, the reaction mixture was diluted with EtOAc and acidified with 1N HCl. The organic layer was washed with brine, dried over Na₂SO₄, filtered and concentrated under reduced pressure. The residue was purified by reverse phase HPLC (C18, 0-100% MeCN in H₂O with 0.1% formic acid) to afford the title compound (24 mg, 41% yield) as a white powder. ¹H NMR (400 MHz, CDCl₃) δ 7.47 (m, 3H), 7.34 (m, 1H), 7.29 (m, 3H), 7.19 (m, 1H), 5.62 (s, 1H), 5.00 (d, J=11.1 Hz, 2H), 4.73 (m, 2H), 3.36 (m, 3H), 2.26 (m, 9H), 1.25 (d, J=7.2 Hz, 9H). LC/MS (m/z) ES+=547.6 (M+1).

### Example 13

### (S)-2-(tert-butoxy)-2-(6-(2-cyclohexyl-N-methylacetamido)-2-(3-fluorobenzoyl)-4,7-dimethylisoindolin-5-yl)acetic acid

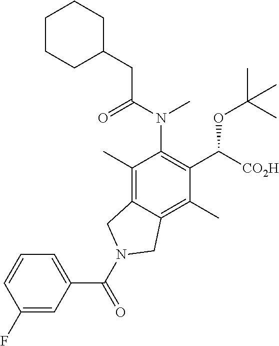 [SMILES: Cc1c2c(c(C)c(N(C)C(=O)CC3CCCCC3)c1[C@H](OC(C)(C)C)C(=O)O)CN(C(=O)c1cccc(F)c1)C2]

The title compound was made in a similar manner as Example 12. ¹H NMR (400 MHz, DMSO) δ 12.07 (br. 1 H), 7.54 (m, 1H), 7.46 (d, J=6.9 Hz, 2H), 7.35 (t, J=8.6 Hz, 1H), 5.13 (d, J=6.0 Hz, 1H), 4.80 (m, 4H), 2.97 (d, J=11.6 Hz, 3H), 2.31 (m, 5H), 1.94 (d, J=63.7 Hz, 3H), 1.60 (m, 5H), 1.17 (m, 12H), 0.84 (m, 3H). LC/MS (m/z) ES+=553.7 (M+1).

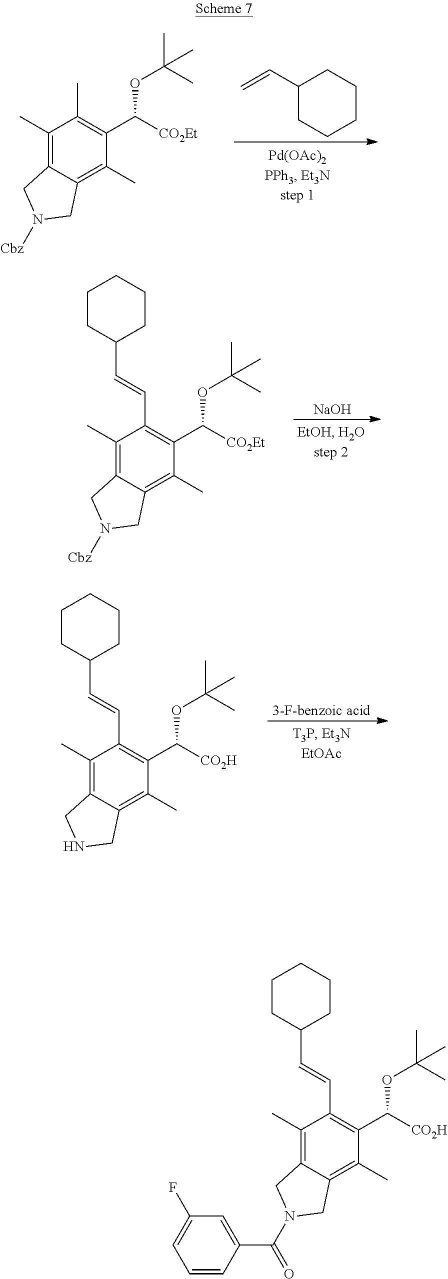

### Example 14

### (S,E)-2-(tert-butoxy)-2-(6-(2-cyclohexylvinyl)-2-(3-fluorobenzoyl)-4,7-dimethylisoindolin-5-yl)acetic acid

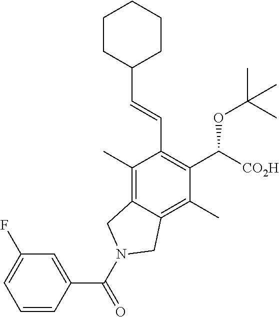 [SMILES: Cc1c(/C=C/C2CCCCC2)c([C@H](OC(C)(C)C)C(=O)O)c(C)c2c1CN(C(=O)c1cccc(F)c1)C2]

### Step 1: (S, E)-benzyl 5-(1-(tert-butoxy)-2-ethoxy-2-oxoethyl)-6-(2-cyclohexylvinyl)-4,7-dimethylisoindoline-2-carboxylate

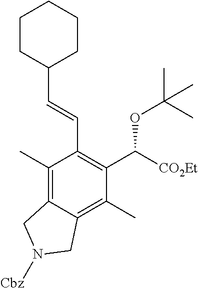 [SMILES: CCOC(=O)[C@@H](OC(C)(C)C)c1c(C)c2c(c(C)c1/C=C/C1CCCCC1)CN(C(=O)OCc1ccccc1)C2]

A mixture of benzyl (S)-5-(1-(tert-butoxy)-2-ethoxy-2-oxoethyl)-6-iodo-4,7-dimethylisoindoline-2-carboxylate (300 mg, 0.53 mmol), vinylcyclohexane (292 mg, 2.7 mmol), Pd(OAc)₂ (24 mg, 0.11 mmol) and PPh₃ (56 mg, 0.21 mmol) in Et₃N (3 mL) was stirred at 100° C. After 18 h, the reaction mixture was partitioned between EtOAc and H₂O. The organic layer was washed with brine, dried over Na₂SO₄, filtered and concentrated under reduced pressure. The residue was purified by reverse phase HPLC (C18, 65-100% MeCN in H₂O with 0.1% formic acid) to afford the title compound (12 mg, 4% yield) as a yellow oil. ¹H NMR (400 MHz, CDCl₃) δ 7.43-7.33 (m, 5H), 6.40 (d, J=16.2 Hz, 1 H), 5.72-5.63 (m, 1H), 5.59 (s, 1H), 5.22 (s, 2H), 4.69 (d, J=16.7 Hz, 4H), 4.24-4.06 (m, 2H), 2.27-2.09 (m, 7H), 1.91-1.76 (m, 4H), 1.25-1.10 (m, 18H). LC/MS (m/z) ES+=548.8 (M+1).

### Step 2: (S,E)-2-(tert-butoxy)-2-(6-(2-cyclohexylvinyl)-4,7-dimethylisoindolin-5-yl)acetic acid

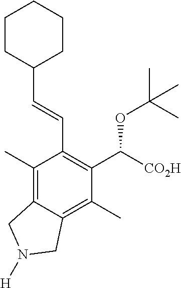 [SMILES: Cc1c(/C=C/C2CCCCC2)c([C@H](OC(C)(C)C)C(=O)O)c(C)c2c1CNC2]

A mixture of (S,E)-benzyl 5-(1-(tert-butoxy)-2-ethoxy-2-oxoethyl)-6-(2-cyclohexylvinyl)-4,7-dimethylisoindoline-2-carboxylate (12 mg, 0.02 mmol) in NaOH (0.15 mL, 5 N) and EtOH (0.3 mL) was stirred at 100° C. After 18 h, the reaction mixture was cooled to ambient temperature and was neutralized with 1N HCl and extracted with DCM/i-PrOH (85:15). The organic layer was washed with brine, dried over Na₂SO₄, filtered and concentrated under reduced pressure to give title compound (10 mg, quant. yield) which was used in the next step without further purification. LC/MS (m/z) ES+=386.4 (M+1).

### Step 3: (S,E)-2-(tert-butoxy)-2-(6-(2-cyclohexylvinyl)-2-(3-fluorobenzoyl)-4,7-dimethylisoindolin-5-yl)acetic acid

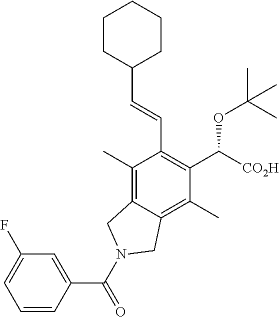 [SMILES: Cc1c(/C=C/C2CCCCC2)c([C@H](OC(C)(C)C)C(=O)O)c(C)c2c1CN(C(=O)c1cccc(F)c1)C2]

To a solution of (S,E)-2-(tert-butoxy)-2-(6-(2-cyclohexylvinyl)-4,7-dimethylisoindolin-5-yl)acetic acid (10 mg, 0.026 mmol) and 3-fluorobenzoic acid (7.3 mg, 0.05 mmol) in EtOAc (0.5 mL) and DCM (1 mL) was added propane phosphonic acid anhyrdide (41 mg, 0.065 mmol, 50% EtOAc solution) and Et₃N (16 mg, 0.16 mmol). After 1 h, the reaction mixture was quenched with sat. aq. NaHCO₃ and extracted with DCM. The organic layer was washed with brine, dried over Na₂SO4, filtered and concentrated under reduced pressure. The residue was purified by reverse phase HPLC (C18, 60-100% MeCN in H₂O with 0.1% formic acid) to afford the title compound (5.5 mg, 42% yield) as a white powder. ¹H NMR (400 MHz, DMSO) δ 12.32 (br, 1H), 7.54 (m, 1H), 7.45 (m, 2H), 7.34 (t, J=8.7 Hz, 1H), 6.43 (m, 1H), 5.66 (m, 1H), 5.47 (d, J=5.2 Hz, 1H), 4.77 (m, 4H), 2.10 (m, 6H), 1.72 (m, 5H), 1.19 (m, 15H). LC/MS (m/z) ES+=508.6 (M+1).

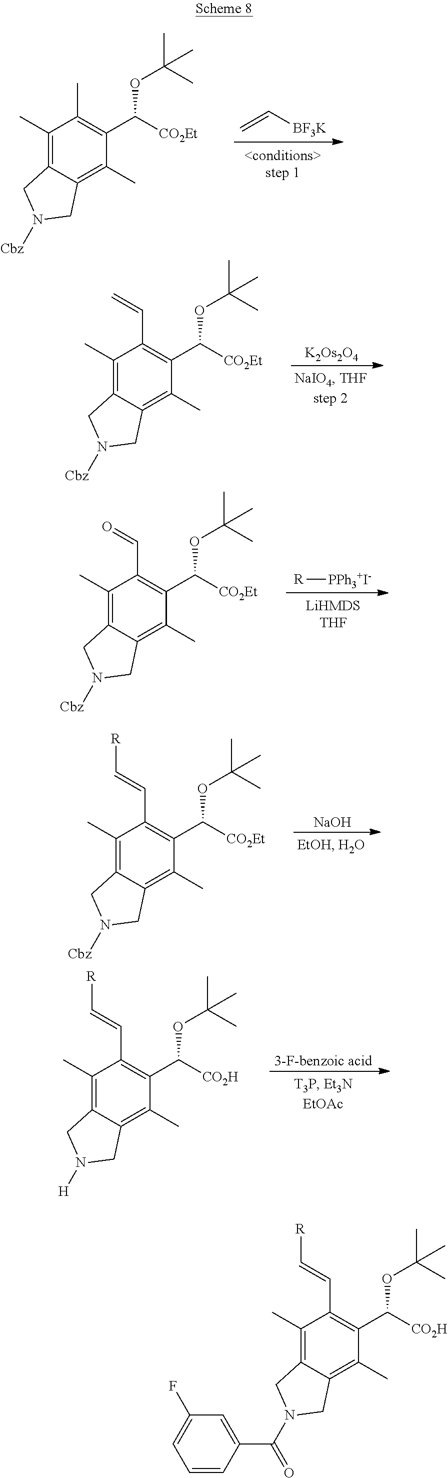

### Example 15

### (S,E)-2-(tert-butoxy)-2-(2-(3-fluorobenzoyl)-4,7-dimethyl-6-(2-(tetrahydro-2H-pran-4-yl)vinyl)isoindolin-5-yl)acetic acid

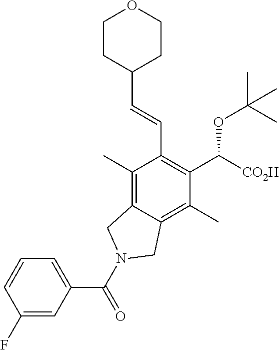 [SMILES: Cc1c(/C=C/C2CCOCC2)c([C@H](OC(C)(C)C)C(=O)O)c(C)c2c1CN(C(=O)c1cccc(F)c1)C2]

### Triphenyl((tetrahydro-2H-pyran-4-yl)methyl)phosphonium

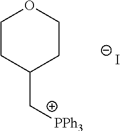

The title compound was prepared from the known procedure as described in J. Med. Chem. 2008, 51, 4340-4345 and references therein.

### Step 1: (S)-benzyl 5-(1-(tert-butoxy)-2-ethoxy-2-oxoethyl)-4,7-dimethyl-6-vinylisoindoline-2-carboxylate

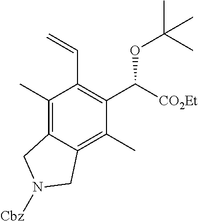 [SMILES: C=Cc1c(C)c2c(c(C)c1[C@H](OC(C)(C)C)C(=O)OCC)CN(C(=O)OCc1ccccc1)C2]

A mixture of benzyl (S)-5-(1-(tert-butoxy)-2-ethoxy-2-oxoethyl)-6-iodo-4,7-dimethylisoindoline-2-carboxylate (10 g, 17.7 mmol), vinylBF₃K (3.8 g, 28.3 mmol), Pd₂(dba)₃ (3.2 g, 3.54 mmol), MePhos (1.3 g, 3.54 mmol) and K₃PO₄ (11 g, 53.1 mmol) in DMF (110 mL) was stirred at 80° C. After 18 h, the reaction mixture was filtered through a pad of Celite and the filtrate was partitioned between EtOAc and H₂O. The organic layer was washed with brine, dried over Na₂SO₄, filtered and concentrated under reduced pressure. The residue was purified by silica gel chromatography (0-30% EtOAc in PE) to afford the title compound (6.1 g, 67% yield) as a yellow solid. LC/MS (m/z) ES+=466.4 (M+1).

### Step 2: (S)-benzyl 5-(1-(tert-butoxy)-2-ethoxy-2-oxoethyl)-6-formyl-4,7-dimethylisoindoline-2-carboxylate

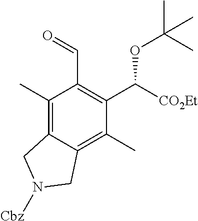 [SMILES: CCOC(=O)[C@@H](OC(C)(C)C)c1c(C)c2c(c(C)c1C=O)CN(C(=O)OCc1ccccc1)C2]

A mixture of benzyl (S)-5-(1-(tert-butoxy)-2-ethoxy-2-oxoethyl)-4,7-dimethyl-6-vinylisoindoline-2-carboxylate (300 mg, 0.64 mmol), NaIO₄ (413 mg, 1.93 mmol) and K₂Os₂O₄ (24 mg, 0.064 mmol) in THF (4 mL) and H₂O (2 mL) was stirred at ambient temperature. After 18 h, the reaction mixture was quenched with sat. aq. Na₂S₂O₃ and extracted with EtOAc. The organic layer was washed with brine, dried over Na₂SO₄, filtered and concentrated under reduced pressure. The residue was purified by silica gel chromatography (0-40% EtOAc in PE) to afford the title compound (170 mg, 56% yield) as a yellow oil. LC/MS (m/z) ES+=490.6 (M+Na).

### Step 3: (S,E)-benzyl 5-(1-(tert-butoxy)-2-ethoxy-2-oxoethyl)-4,7-dimethyl-6-(2-(tetrahydro-2H-pyran-4-yl)vinyl)isoindoline-2-carboxylate

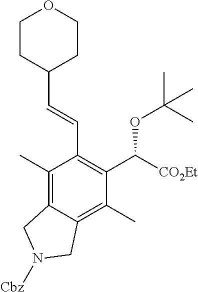 [SMILES: CCOC(=O)[C@@H](OC(C)(C)C)c1c(C)c2c(c(C)c1/C=C/C1CCOCC1)CN(C(=O)OCc1ccccc1)C2]

A −78° C. suspension of triphenyl((tetrahydro-2H-pyran-4-yl)methyl)phosphonium iodide (409 mg, 0.84 mmol) in THF (2 mL) was treated with LiHMDS (0.8 mL, 0.8 mmol, 1.0 M). After 30 min, a solution of benzyl (S)-5-(1-(tert-butoxy)-2-ethoxy-2-oxoethyl)-6-formyl-4,7-dimethylisoindoline-2-carboxylate (98 mg, 0.21 mmol) in THF was added dropwise. The reaction mixture was warmed to ambient temperature and was quenched with sat. aq. NH₄Cl solution and extracted with EtOAc. The organic layer was washed with brine, dried over Na₂SO₄, filtered and concentrated under reduced pressure. The residue was purified by silica gel chromatography (0-30% EtOAc in PE) to afford the title compound (50 mg, 43% yield) as a yellow oil. ¹H NMR (400 MHz, CDCl₃) δ 7.42-7.34 (m, 5H), 6.48 (d, J=16.3 Hz, 1H), 5.72-5.65 (m, 1H), 5.54 (s, 1H), 5.22 (s, 2H), 4.73-4.65 (m, 4H), 4.22-4.09 (m, 2H), 4.07-4.00 (m, 2H), 3.50 (td, J=11.8, 1.2 Hz, 2H), 2.53-2.45 (m, 1H), 2.27 (d, J=10.9 Hz, 3H), 2.11 (d, J=12.3 Hz, 3H), 1.77-1.64 (m, 4H), 1.20-1.11 (m, 12H). LC/MS (m/z) ES+=550.7 (M+1).

### Step 4: (S,E)-2-(tert-butoxy)-2-(4,7-dimethyl-6-(2-(tetrahydro-2H-pyran-4-yl)vinyl)isoindolin-5-yl)acetic acid

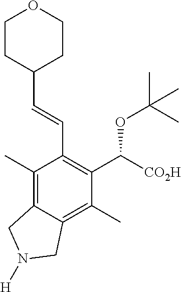 [SMILES: Cc1c(/C=C/C2CCOCC2)c([C@H](OC(C)(C)C)C(=O)O)c(C)c2c1CNC2]

A mixture of (S,E)-benzyl 5-(1-(tert-butoxy)-2-ethoxy-2-oxoethyl)-6-(2-cyclohexylvinyl)-4,7-dimethylisoindoline-2-carboxylate (50 mg, 0.09 mmol) and NaOH (108 mg, 2.7 mmol) in EtOH (1.1 mL) and H₂O (0.55 mL) was stirred at 100° C. under N₂ atmosphere overnight. After cooled down to r.t., the resulting mixture was neutralized with 1N HCl and extracted with DCM/i-PrOH (85:15). The organic layer was washed with brine, dried over Na₂SO₄, filtered and concentrated under reduced pressure to give title compound (31 mg, 89% yield) which was used in the next step without further purification. LC/MS (m/z) ES+=388.3 (M+1).

### Step 5: (S,E)-2-(tert-butoxy)-2-(2-(3-fluorobenzoyl)-4,7-dimethyl-6-(2-(tetrahydro-2H-pyran-4-yl)vinyl)isoindolin-5-yl)acetic acid

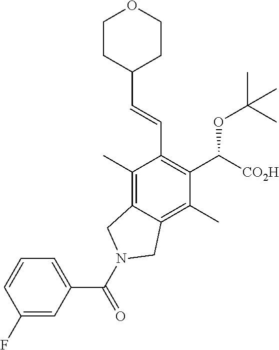 [SMILES: Cc1c(/C=C/C2CCOCC2)c([C@H](OC(C)(C)C)C(=O)O)c(C)c2c1CN(C(=O)c1cccc(F)c1)C2]

A solution of (S,E)-2-(tert-butoxy)-2-(4,7-dimethyl-6-(2-(tetrahydro-2H-pyran-4-yl)vinyl)iso indolin-5-yl)acetic acid (31 mg, 0.08 mmol) and 3-fluorobenzoic acid (22 mg, 0.16 mmol) in EtOAc (2 mL) and DCM (2 mL) was added propane phosphonic acid anhyrdide (127 mg, 0.2 mmol, 50% EtOAc solution) and Et₃N (29 mg, 0.48 mmol). After 1 h, the resulting mixture was quenched with sat. NaHCO₃ aq. solution and extracted with DCM. The organic layer was washed with brine, dried over Na₂SO₄, filtered and concentrated under reduced pressure. The residue was purified by reverse phase HPLC (C18, 50-100% MeCN in H₂O with 0.1% formic acid) to afford the title compound (10 mg, 24% yield) as a white powder. ¹H NMR (400 MHz, DMSO) δ 12.55 (br, 1H), 7.59-7.50 (m, 1H), 7.49-7.40 (m, 2H), 7.34 (t, J=8.8 Hz, 1H), 6.49 (dd, J=15.9, 13.0 Hz, 1H), 5.76-5.62 (m, 1H), 5.42 (d, J=5.6 Hz, 1 H), 4.90-4.57 (m, 4H), 3.89 (dd, J=9.3, 4.7 Hz, 2H), 3.45-3.36 (m, 3H), 2.30-1.94 (m, 6H), 1.78-1.61 (m, 2H), 1.50-1.35 (m, 2H), 1.05 (d, J=7.7 Hz, 9H). LC/MS (m/z) ES+=510.2 (M+1).

### Example 16

### (S,E)-2-(tert-butoxy)-2-(6-(4,4-dimethylpent-1-en-1-yl)-2-(3-fluorobenzoyl)-4,7-dimethylisoindolin-5-yl)acetic acid

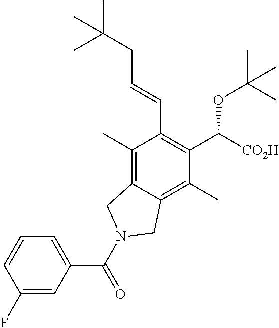 [SMILES: Cc1c(/C=C/CC(C)(C)C)c([C@H](OC(C)(C)C)C(=O)O)c(C)c2c1CN(C(=O)c1cccc(F)c1)C2]

The title compound was made in a similar manner as Example 15 except using (3,3-Dimethylbutyl)triphenylphosphonium (WO200463179A1) in Step 3. ¹H NMR (400 MHz, CDCl₃) δ 9.79 (br, 1H), 7.49-7.41 (m, 1 H), 7.35 (d, J=7.7 Hz, 1H), 7.29-7.26 (m, 1H), 7.22-7.15 (m, 1H), 6.50-6.41 (m, 1H), 5.96-5.83 (m, 1H), 5.75 (s, 1H), 5.03-4.88 (m, 2H), 4.74-4.64 (m, 2H), 2.29-2.03 (m, 8H), 1.14 (d, J=7.6 Hz, 9H), 0.96 (d, J=7.6 Hz, 9H). LC/MS (m/z) ES+=496.2 (M+1).

### Example 17

### (S,E)-2-(tert-butoxy)-2-(2-(3-fluorobenzoyl)-4,7-dimethyl-6-styrylisoindolin-5-yl)acetic acid

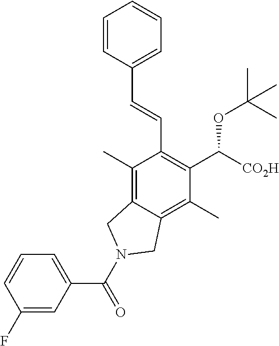 [SMILES: Cc1c(/C=C/c2ccccc2)c([C@H](OC(C)(C)C)C(=O)O)c(C)c2c1CN(C(=O)c1cccc(F)c1)C2]

The title compound was made in a similar manner as Example 15 except using Benzyltriphenylphosphonium bromide in Step 3. ¹H NMR (400 MHz, DMSO) δ 12.41 (s, 1H), 7.42 (m, 10H), 6.62 (m, 1H), 5.48 (d, J=6.7 Hz, 1H), 4.82 (m, 4H), 2.18 (m, 6H), 1.05 (t, J=8.9 Hz, 9H). LC/MS (m/z) ES+=502.5 (M+1).

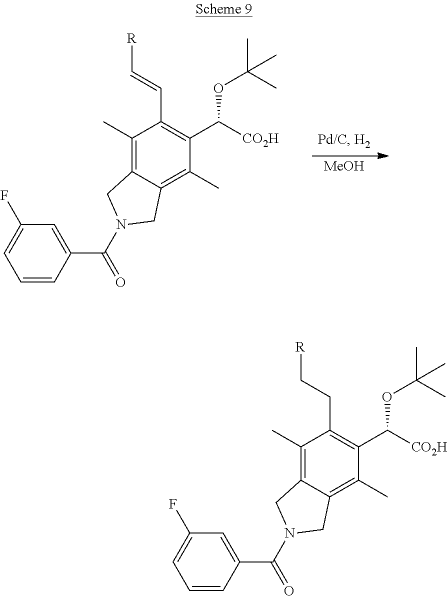

### Example 18

### (S)-2-(tert-butoxy)-2-(2-(3-fluorobenzoyl)-4,7-dimethyl-6-phenethylisoindolin-5-yl)acetic acid

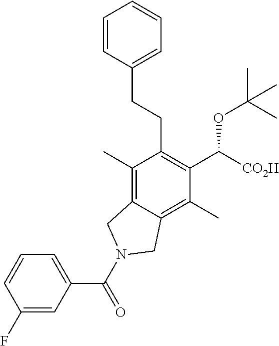 [SMILES: Cc1c2c(c(C)c([C@H](OC(C)(C)C)C(=O)O)c1CCc1ccccc1)CN(C(=O)c1cccc(F)c1)C2]

A mixture of (S,E)-2-(tert-butoxy)-2-(2-(3-fluorobenzoyI)-4,7-dimethyl-6-styrylisoindolin-5-yl)acetic acid (20 mg, 0.04 mmol) and 10% Pd/C (20 mg) in MeOH (1 mL) was hydrogenated under H₂ atmosphere (1 atm). After 1 h, the resulting mixture was filtered through a pad of Celite and the residue was concentrated under reduced pressure. The residue was purified by reverse phase HPLC to afford the title compound (12 mg, 60% yield) as a white powder. ¹H NMR (400 MHz, DMSO) δ 12.51 (br, 1H), 7.55 (m, 1H), 7.46 (d, J=7.7 Hz, 2H), 7.35 (m, 5H), 7.21 (m, 1 H), 5.41 (d, J=17.2 Hz, 1 H), 4.79 (m, 4H), 2.98 (m, 4H), 2.24 (m, 6H), 1.19 (d, J=8.3 Hz, 9H). LC/MS (m/z) ES+=504.5 (M+1).

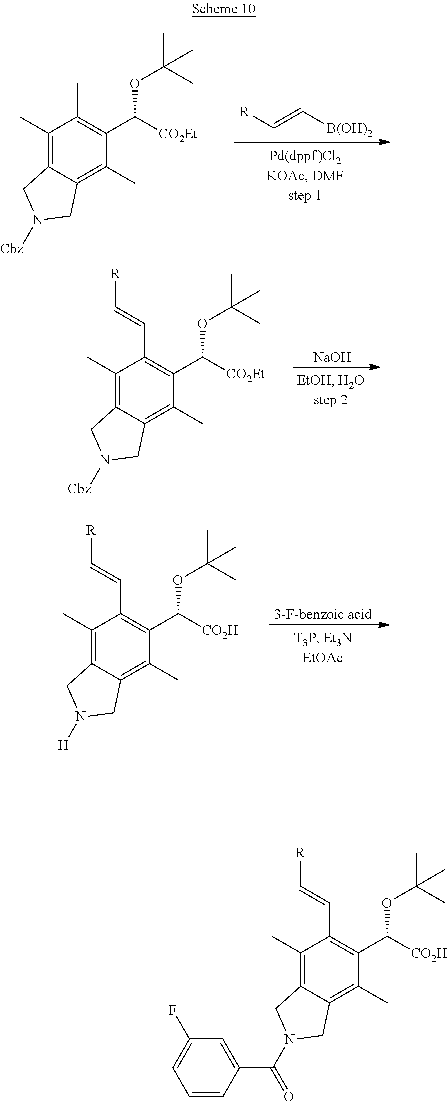

### Example 19

### (S,E)-2-(tert-butoxy)-2-(2-(3-fluorobenzoyl)-4,7-dimethyl-6-(4-methylstyryl)isoindolin-5-yl)acetic acid

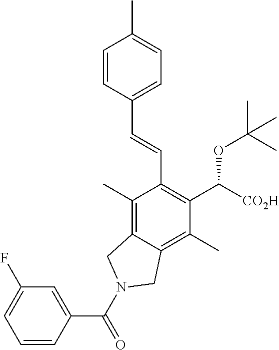 [SMILES: Cc1ccc(/C=C/c2c(C)c3c(c(C)c2[C@H](OC(C)(C)C)C(=O)O)CN(C(=O)c2cccc(F)c2)C3)cc1]

### Step 1: (S,E)-benzyl 5-(1-(tert-butoxy)-2-ethoxy-2-oxoethyl)-4,7-dimethyl-6-(4-methylstyryl)isoindoline-2-carboxylate

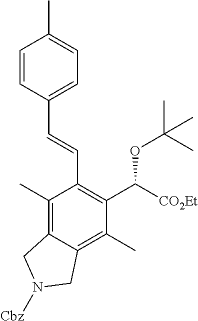 [SMILES: CCOC(=O)[C@@H](OC(C)(C)C)c1c(C)c2c(c(C)c1/C=C/c1ccc(C)cc1)CN(C(=O)OCc1ccccc1)C2]

A mixture of benzyl (S)-5-(1-(tert-butoxy)-2-ethoxy-2-oxoethyl)-6-iodo-4,7-dimethylisoindoline-2-carboxylate (150 mg, 0.27 mmol), (E)-(4-methylstyryl)boronic acid (87 mg, 0.53 mmol) Pd(dppf)Cl₂ (22 mg, 0.127 mmol), KOAc (80 mg, 0.81 mmol) in DMF (1.5 mL) was stirred at 80° C. After 18 h, the reaction mixture was cooled to ambient temperature and the resulting mixture was partitioned between EtOAc and H₂O. The layers were separated and the organic layer was washed with brine, dried over Na₂SO₄, filtered and concentrated under reduced pressure. The residue was purified by silica gel chromatography (silica gel, 0-20% EtOAc in PE) to afford the title compound (120 mg, 82% yield) as a yellow solid. LC/MS (m/z) ES+=556.4 (M+1).

### Step 2: (S,E)-2-(tert-butoxy)-2-(4,7-dimethyl-6-(4-methylstyryl)isoindolin-5-yl)acetic acid

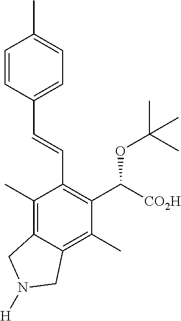 [SMILES: Cc1ccc(/C=C/c2c(C)c3c(c(C)c2[C@H](OC(C)(C)C)C(=O)O)CNC3)cc1]

A mixture of benzyl (S,E)-5-(1-(tert-butoxy)-2-ethoxy-2-oxoethyl)-4,7-dimethyl-6-(4-methylstyryl) isoindoline-2-carboxylate (160 mg, 0.29 mmol) and NaOH (346 mg, 8.64 mmol) in EtOH (3 mL) and H₂O (1.5 mL) was stirred at 100° C. After 18 h, the reaction mixture was cooled to ambient temperature and acidified with 6 N HCl and extracted with DCM/i-PrOH (85:15). The organic layer was washed with brine, dried over Na₂SO₄, filtered and concentrated under reduced pressure to give the title compound (110 mg, quant. yield) as a brown oil which was used in the next step without further purification. LC/MS (m/z) ES+=394.4 (M+1).

### Step 3. (S,E)-2-(tert-butoxy)-2-(2-(3-fluorobenzoyl)-4,7-dimethyl-6-(4-methylstyryl)isoindolin-5-yl)acetic acid

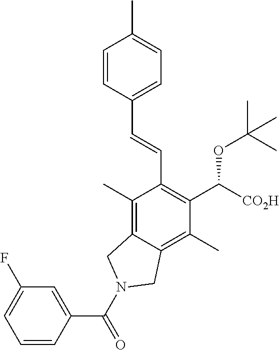 [SMILES: Cc1ccc(/C=C/c2c(C)c3c(c(C)c2[C@H](OC(C)(C)C)C(=O)O)CN(C(=O)c2cccc(F)c2)C3)cc1]

A solution of (S,E)-2-(tert-butoxy)-2-(4,7-dimethyl-6-(4-methylstyryl)isoindolin-5-yl)acetic acid (115 mg, 0.29 mmol) and 3-fluorobenzoic acid (82 mg, 0.58 mmol) in DCM (3 mL) was treated with propane phosphonic acid anhyrdide (464 mg, 0.73 mmol, 50% EtOAc solution) and Et₃N (118 mg, 1.17 mmol). After 30 min, the resulting mixture was quenched with sat. aq. NaHCO₃ and extracted with DCM. The organic layer was washed with half sat. aq. citric acid and brine, dried over Na₂SO₄, filtered and concentrated under reduced pressure. The residue was purified by reverse phase HPLC (C18, 50-100% MeCN in H₂O with 0.1% formic acid) to afford the title compound (70 mg, 47% yield) as a white powder. ¹H NMR (400 MHz, DMSO) δ 12.46 (br, 1H), 7.55 (dd, J=13.9, 7.6 Hz, 1H), 7.46 (m, 4H), 7.32 (m, 2H), 7.21 (m, 2H), 6.57 (m, 1 H), 5.49 (d, J=6.4 Hz, 1 H), 4.79 (m, 4H), 2.22 (m, 9H), 1.03 (d, J=7.5 Hz, 9H). LC/MS (m/z) ES+=516.6 (M+1).

### Example 20

### (S)-2-(tert-butoxy)-2-(2-(3-fluorobenzoyl)-4,7-dimethyl-6-(4-methylphenethyl)isoindolin-5-yl)acetic acid

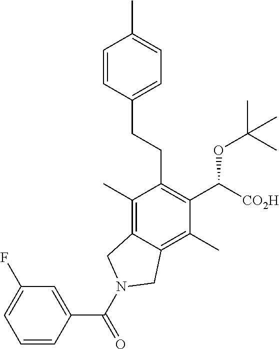 [SMILES: Cc1ccc(CCc2c(C)c3c(c(C)c2[C@H](OC(C)(C)C)C(=O)O)CN(C(=O)c2cccc(F)c2)C3)cc1]

The title compound was made in a similar manner as Example 18. ¹H NMR (400 MHz, DMSO) δ 12.44 (br, 1H), 7.55 (m, 1H), 7.46 (d, J=7.7 Hz, 2H), 7.35 (t, J=8.7 Hz, 1H), 7.21 (d, J=8.2 Hz, 2H), 7.12 (t, J=7.0 Hz, 2H), 5.41 (d, J=17.3 Hz, 1H), 4.79 (d, J=36.1 Hz, 4H), 2.86 (m, 4H), 2.26 (m, 9H), 1.20 (d, J=12.4 Hz, 9H). LC/MS (m/z) ES+=518.5 (M+1).

### Example 21

### (S,E)-2-(tert-butoxy)-2-(2-(3-fluorobenzoyl)-4,7-dimethyl-6-(2-phenylprop-1-en-1-yl)isoindolin-5-yl)acetic acid

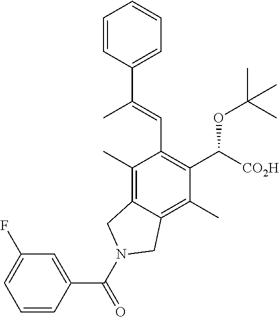 [SMILES: C/C(=C\c1c(C)c2c(c(C)c1[C@H](OC(C)(C)C)C(=O)O)CN(C(=O)c1cccc(F)c1)C2)c1ccccc1]

The title compound was made in a similar manner as Example 14 except using prop-1-en-2-ylbenzene in Step 1. ¹H NMR (400 MHz, DMSO) δ 12.33 (br, 1H), 7.46 (m, 9H), 6.86 (m, 1H), 5.37 (m, 1H), 4.79 (m, 4H), 2.08 (m, 9H), 1.09 (m, 9H). LC/MS (m/z) ES+=516.6 (M+1).

### Example 22

### (S,E)-2-(tert-butoxy)-2-(2-(3-fluorobenzoyl)-4,7-dimethyl-6-(1-phenylprop-1-en-2-yl)isoindolin-5-yl)acetic acid

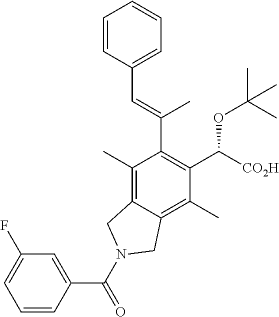 [SMILES: C/C(=C\c1ccccc1)c1c(C)c2c(c(C)c1[C@H](OC(C)(C)C)C(=O)O)CN(C(=O)c1cccc(F)c1)C2]

The title compound was made in a similar manner as Example 19 except using (Z)-4,4,5,5-tetramethyl-2-(1-phenylprop-1-en-2-yl)-1,3,2-dioxaborolane (J. Org. Chem. 2013, 78, 12837) in Step 1. ¹H NMR (400 MHz, DMSO) δ 12.37 (br, 1H), 7.41 (m, 9H), 6.24 (d, J=11.8 Hz, 1H), 5.30 (d, J=4.9 Hz, 1 H), 4.79 (m, 4H), 2.15 (m, 9H), 1.12 (d, J=8.7 Hz, 9H). LC/MS (m/z) ES+=516.6 (M+1).

### Example 23

### (S,E)-2-(tert-butoxy)-2-(2-(3-fluorobenzoyl)-4,7-dimethyl-6-(3-phenylbut-2-en-2-yl)isoindolin-5-yl)acetic acid

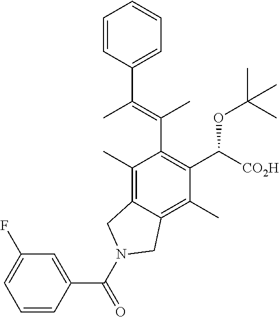 [SMILES: C/C(=C(/C)c1c(C)c2c(c(C)c1[C@H](OC(C)(C)C)C(=O)O)CN(C(=O)c1cccc(F)c1)C2)c1ccccc1]

The title compound was made in a similar manner as Example 19 except using (Z)-4,4,5,5-tetramethyl-2-(3-phenylbut-2-en-2-yl)-1,3,2-dioxaborolane (J. Am. Chem. Soc. 2012, 134, 15168) in Step 1. ¹H NMR (400 MHz, DMSO) δ 12.38 (br, 1H), 7.41 (m, 9H), 5.33 (dd, J=18.8, 4.2 Hz, 1H), 4.79 (m, 4H), 2.20 (m, 6H), 1.67 (m, 6H), 1.21 (m, 9H). LC/MS (m/z) ES+=530.6 (M+1).

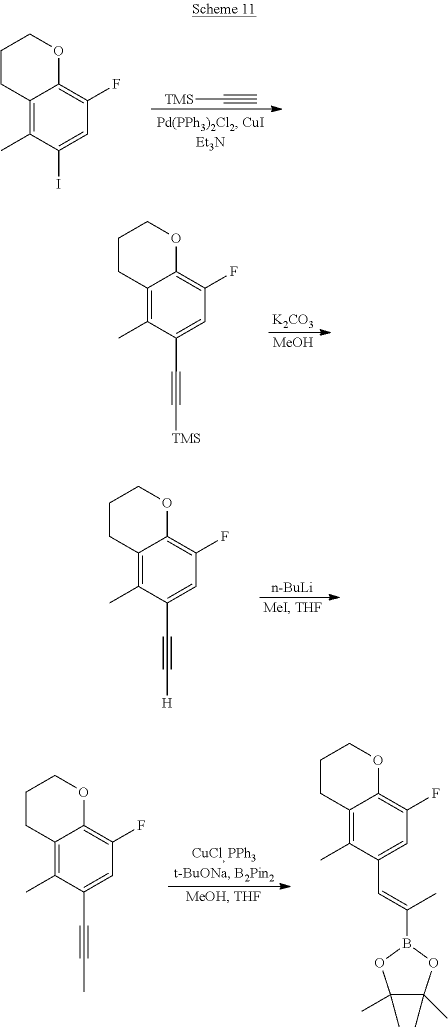

### (Z)-2-(1-(8-fluoro-5-methylchroman-6-yl)prop-1-en-2-yl)-4,4,5,5-tetramethyl-1,3,2-dioxaborolane

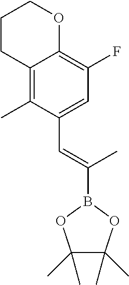 [SMILES: C/C(=C\c1cc(F)c2c(c1C)CCCO2)B1OC(C)(C)C(C)(C)O1]

### Step 1. ((8-fluoro-5-methylchroman-6-yl)ethynyl)trimethylsilane

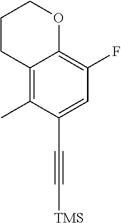 [SMILES: Cc1c(C#C[Si](C)(C)C)cc(F)c2c1CCCO2]

A mixture of 8-fluoro-6-iodo-5-methylchromane (2.5 g, 8.59 mmol), ethynyltrimethylsilane (4.2 g, 43 mmol), Pd(dppf)Cl₂ (600 mg, 0.859 mmol) and CuI (326 mg, 1.72 mmol) in Et₃N (20 mL) was stirred at ambient temperature. After 18 h, the reaction mixture was filtered through a pad of Celite and the filtrate was concentrated under reduced pressure. The residue was purified by silica gel chromatography ISCO (0-5% EtOAc in PE) to afford the title compound (2.0 g, 90% yield) as a yellow solid. LC/MS (m/z) ES+=263.1 (M+1).

### Step 2. 6-ethynyl-8-fluoro-5-methylchroman

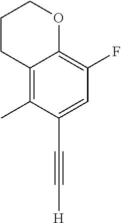

A solution of ((8-fluoro-5-methylchroman-6-yl)ethynyl)trimethylsilane (2.0 g, 8 mmol) in MeOH (20 mL) was treated with K₂CO₃ (2.2 g, 16 mmol). After 3 h, the reaction mixture was filtered and the filtrate concentrated in vacuo. The residue was purified by silica gel chromatography (0-10% EtOAc in PE) to afford the title compound (1.2 g, 75% yield) as a white solid. ¹H NMR (400 MHz, CDCl₃) δ 7.05 (d, J=11.2 Hz, 1H), 4.26-4.13 (m, 2H), 3.18 (s, 1H), 2.66 (t, J=6.6 Hz, 2H), 2.30 (d, J=0.6 Hz, 3H), 2.17-2.02 (m, 2H). LC/MS (m/z) ES+=191.4 (M+1).

### Step 3. 8-fluoro-5-methyl-6-(prop-1-yn-1-yl)chroman

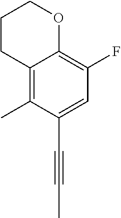

A −30° C. solution of 6-ethynyl-8-fluoro-5-methylchromane (600 mg, 3.15 mmol) in THF (8 mL) was treated with n-BuLi (2.5 M, 1.86 mL, 4.7 mmol). After 30 min, iodomethane was added and the reaction mixture was warmed to ambient temperature. After 2 h, the reaction mixture was quenched with sat. aq. NH₄Cl solution and extracted with EtOAc. The organic layer was washed with brine, dried over Na₂SO₄, filtered and concentrated under reduced pressure. The residue was purified by silica gel chromatography (0-10% EtOAc in PE) to afford the title compound (560 mg, 87% yield) as a white solid. LC/MS (m/z) ES+=205.3 (M+1).

### Step 4. (Z)-2-(1-(8-fluoro-5-methylchroman-6-yl)prop-1-en-2-yl)-4,4,5,5-tetramethyl-1,3,2-dioxaborolane

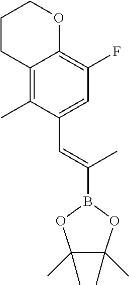 [SMILES: C/C(=C\c1cc(F)c2c(c1C)CCCO2)B1OC(C)(C)C(C)(C)O1]

A mixture of CuCl (20 mg, 0.196 mmol), PPh₃ (51.5 mg, 0.196 mmol) and t-BuONa (226 mg, 2.35 mmol) in THF was stirred at ambient temperature. After 30 min, a solution of 8-fluoro-5-methyl-6-(prop-1-yn-1-yl)chromane (400 mg, 1.96 mmol) was added, followed by the addition of MeOH (157 mg, 3.92 mmol). After 18 h, the resulting mixture was partitioned between EtOAc and H₂O. The organic layer was washed with brine, dried over Na₂SO₄, filtered and concentrated under reduced pressure. The residue was purified by silica gel chromatography (silica gel, 0-5% EtOAc in PE) to afford the title compound (200 mg, 31% yield) as a white solid. LC/MS (m/z) ES+=333.4 (M+1).

### Example 24

### (S,E)-2-(tert-butoxy)-2-(6-(1-(8-fluoro-5-methylchroman-6-yl)prop-1-en-2-yl)-2-(3-fluorobenzoyl)-4,7-dimethylisoindolin-5-yl)acetic acid

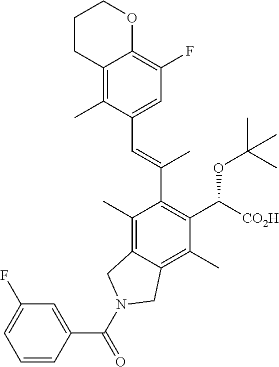 [SMILES: C/C(=C\c1cc(F)c2c(c1C)CCCO2)c1c(C)c2c(c(C)c1[C@H](OC(C)(C)C)C(=O)O)CN(C(=O)c1cccc(F)c1)C2]

The title compound was made in a similar manner as Example 19 except using (Z)-2-(1-(8-fluoro-5-methylchroman-6-yl)prop-1-en-2-yl)-4,4,5,5-tetramethyl-1,3,2-dioxaborolane in Step 1. ¹H NMR (400 MHz, CDCl₃) δ 9.80 (br, 1H), 7.46 (dd, J=13.6, 7.8 Hz, 1H), 7.35 (d, J=7.6 Hz, 1H), 7.31-7.26 (m, 1H), 7.19 (t, J=8.4 Hz, 1H), 6.89 (dd, J=20.1, 8.3 Hz, 1H), 6.29 (s, 1H), 5.52 (s, 1H), 5.10-4.87 (m, 2H), 4.71 (d, J=7.0 Hz, 2H), 4.22 (dd, J=9.7, 4.6 Hz, 2H), 2.69 (dd, J=11.3, 5.6 Hz, 2H), 2.38-2.03 (m, 14H), 1.40-1.11 (m, 9H). LC/MS (m/z) ES+=604.7 (M+1).

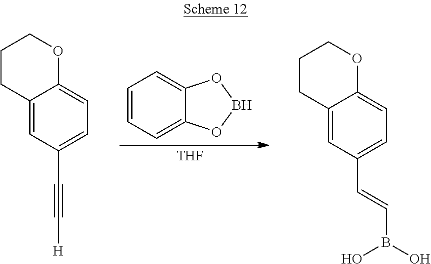

### (E)-(2-(chroman-6-yl)vinyl)boronic acid

A solution of 6-ethynylchromane (200 mg, 1.27 mmol) (made according to WO200876043/A1) in THF (2.5 mL) was treated with catacolborane (303 mg, 2.53 mmol) and heated to 70° C. After 1.5 h, the reaction mixture was quenched with MeOH (1 mL) and partitioned between EtOAc and H₂O. The organic layer was washed with brine, dried over Na₂SO₄, filtered and concentrated under reduced pressure. The residue was purified by silica gel chromatography (0-10% EtOAc in PE) to afford the title compound (90 mg, 37% yield) as a white solid. ¹H NMR (400 MHz, CDCl₃) δ 7.17 (dd, J=8.5, 2.0 Hz, 1H), 7.14-7.09 (m, 2H), 6.76 (d, J=8.4 Hz, 1 H), 4.22-4.14 (m, 2H), 2.78 (t, J=6.5 Hz, 2H), 2.03-1.97 (m, 5H), 1.56 (s, 3H), 1.30 (s, 12H). LC/MS (m/z) ES−=249.3 (M+HCOOH-1).

### Example 25

### (S,E)-2-(tert-butoxy)-2-(6-(2-(chroman-6-yl)vinyl)-2-(3-fluorobenzoyl)-4,7-dimethylisoindolin-5-yl)acetic acid

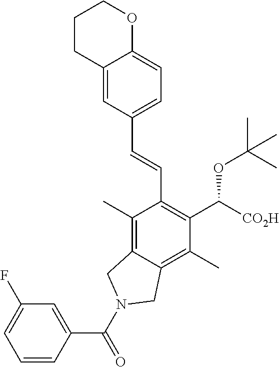 [SMILES: Cc1c(/C=C/c2ccc3c(c2)CCCO3)c([C@H](OC(C)(C)C)C(=O)O)c(C)c2c1CN(C(=O)c1cccc(F)c1)C2]

The title compound was made in a similar manner as Example 19 except using (E)-(2-(chroman-6-yl)vinyl)boronic acid in Step 1. ¹H NMR (400 MHz, CDCl₃) δ 9.75 (br, 1H), 7.50-7.40 (m, 1H), 7.36 (d, J=7.3 Hz, 1H), 7.29 (d, J=4.9 Hz, 2H), 7.24-7.11 (m, 2H), 7.01 (dd, J=16.4, 12.4 Hz, 1H), 6.80 (dd, J=8.4, 5.6 Hz, 1H), 6.72-6.53 (m, 1H), 5.77 (s, 1H), 5.09-4.87 (m, 2H), 4.80-4.60 (m, 2H), 4.21 (dd, J=9.8, 4.2 Hz, 2H), 2.81 (dd, J=11.8, 6.0 Hz, 2H), 2.35-1.98 (m, 8H), 1.12 (d, J=8.1 Hz, 9H). LC/MS (m/z) ES+=558.6 (M+1).

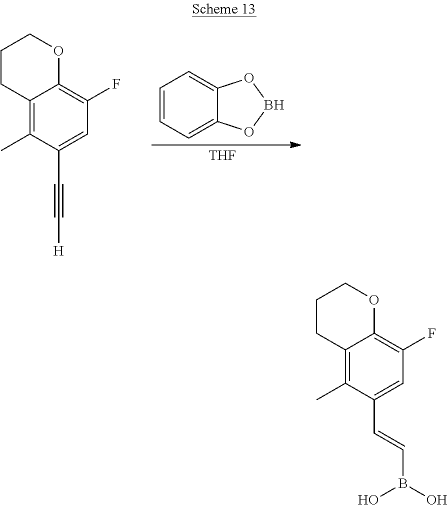

### (E)-(2-(8-fluoro-5-methylchroman-6-yl)vinyl)boronic acid.

To a solution of 6-ethynyl-8-fluoro-5-methylchromane (200 mg, 1.05 mmol) in THF (3 mL) was added catacolborane (235 mg, 2.10 mmol) and heated to 70° C. After 1.5 h, the resulting mixture was quenched with MeOH (1 mL) and partitioned between EtOAc and H₂O. The organic layer was washed with brine, dried over Na₂SO₄, filtered and concentrated under reduced pressure. The residue was purified by silica gel chromatography (silica gel, 0-10% EtOAc in PE) to afford the title compound (50 mg, 22% yield) as a white solid. LC/MS (m/z) ES−=281.4 (M+HCOOH-1).

### Example 26

### (S)-2-(tert-butoxy)-2-(6-(2-(8-fluoro-5-methylchroman-6-yl)vinyl)-2-(3-fluorobenzoyn-4,7-dimethylisoindolin-5-yl)acetic acid

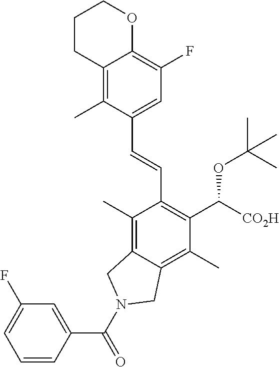 [SMILES: Cc1c(/C=C/c2cc(F)c3c(c2C)CCCO3)c([C@H](OC(C)(C)C)C(=O)O)c(C)c2c1CN(C(=O)c1cccc(F)c1)C2]

The title compound was made in a similar manner as Example 19 except using (E)-(2-(8-fluoro-5-methylchroman-6-yl)vinyl)boronic acid in Step 1. ¹H NMR (400 MHz, CDCl₃) δ 9.80 (br, 1H), 7.51-7.41 (m, 1H), 7.36 (d, J=7.0 Hz, 1H), 7.31-7.28 (m, J=1.5 Hz, 1H), 7.25-7.15 (m, 2H), 7.09-6.84 (m, 2H), 5.78 (s, 1H), 5.12-4.87 (m, 2H), 4.80-4.64 (m, 2H), 4.23 (dd, J=9.7, 4.0 Hz, 2H), 2.70 (dd, J=10.7, 6.4 Hz, 2H), 2.41-1.96 (m, 11H), 1.14 (d, J=6.9 Hz, 9H). LC/MS (m/z) ES+=590.4 (M+1).

### Example 27

### (S,Z)-2-(tert-butoxy)-2-(2-(3-fluorobenzoyl)-4,7-dimethyl-6-styrylisoindolin-5-yl)acetic acid

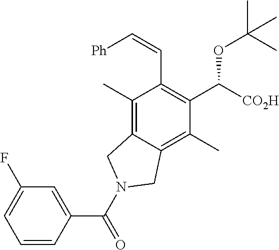 [SMILES: Cc1c(/C=C\c2ccccc2)c([C@H](OC(C)(C)C)C(=O)O)c(C)c2c1CN(C(=O)c1cccc(F)c1)C2]

The title compound was made in a similar manner as Example 19 except using (Z)-styrylboronic acid (J. Am. Chem. Soc. 2015, 137, 3233-3236) in Step 1. ¹H NMR (400 MHz, CDCl₃) δ 9.56 (br, 1H), 7.54-7.26 (m, 4H), 7.23-7.08 (m, 4H), 7.01 (d, J=10.2 Hz, 1H), 6.84-6.50 (m, 2H), 5.63 (d, J=48.8 Hz, 1 H), 5.11-4.61 (m, 4H), 2.43-1.83 (m, 6H), 1.14 (d, J=10.7 Hz, 9H). LC/MS (m/z) ES+=502.1 (M+1).

### Anti-HIV Activity

### MT4 Assay

Antiviral HIV activity and cytotoxicity values for compounds of the invention from Table 1 were measured in parallel in the HTLV-1 transformed cell line MT-4 based on the method previously described (Hazen et al., 2007, In vitro antiviral activity of the novel, tyrosyl-based human immunodeficiency virus (HIV) type 1 protease inhibitor brecanavir (GW640385) in combination with other antiretrovirals and against a panel of protease inhibitor-resistant HIV (Hazen et al., “In vitro antiviral activity of the novel, tyrosyl-based human immunodeficiency virus (HIV) type 1 protease inhibitor brecanavir (GW640385) in combination with other antiretrovirals and against a panel of protease inhibitor-resistant HIV”, Antimicrob. Agents Chemother. 2007, 51: 3147-3154; and Pauwels et al., “Sensitive and rapid assay on MT-4 cells for the detection of antiviral compounds against the AIDS virus”, J. of Virological Methods 1987, 16: 171-185).

Luciferase activity was measured 96 hours later by adding a cell titer glo (Promega, Madison, Wis.). Percent inhibition of cell protection data was plotted relative to no compound control. Under the same condition, cytotoxicity of the compounds was determined using cell titer Glo™ (Promega, Madison, Wis). ICsos were determined from a 10 point dose response curve using 3-4-fold serial dilution for each compound, which spans a concentration range >1000 fold.

These values are plotted against the molar compound concentrations using the standard four parameter logistic equation:

y=((Vmax*x̂n)/(K̂n+x̂n))+Y2

where:

Y2=minimum y n=slope factor

Vmax=maximum y x=compound concentration [M]

K=EC₅₀

When tested in the MT4 assay compounds were found to have IC₅₀ values listed in Table 1.

TABLE 1

| | IC50 |
|---|---|
| Example | (μM) |
| 1 | 1.03 |
| 2 | 3.68 |
| 3 | 0.651 |
| 4 | 10.18 |
| 5 | 50 |
| 6 | 9.63 |
| 7 | 38 |
| 8 | 50 |
| 9 | 50 |
| 10 | 4.10 |
| 11 | 3.12 |
| 12 | 33.9 |
| 13 | 44.64 |
| 14 | 0.086 |
| 15 | 1.31 |
| 16 | 0.120 |
| 17 | 0.124 |
| 18 | 1.09 |
| 19 | 50 |
| 20 | 50 |
| 21 | 0.103 |
| 22 | 0.039 |
| 23 | 0.044 |
| 24 | 50 |
| 25 | 33.9 |
| 26 | 50 |
| 27 | 0.868 |

1. A compounds of Formula I or a pharmaceutically acceptable salt thereof: 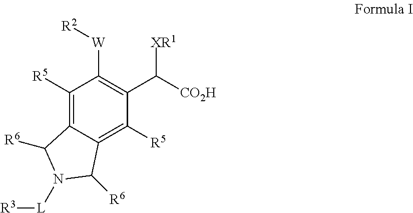
wherein:
X is O or CH₂;
R¹ is C₁₋₆alkyl wherein said alkyl may contain cycloalkyl portions;
W is —CH═CH—, C≡C—, C₁₋₃alkylene, —CH₂C(O)NH—, —NHC(O)—, —N(CH₃)C(O)—, —N(CH₃)C(O)CH₂—, —C(O)—, —CH₂C(O)—, or —NHC(O)CH₂—, wherein each W is optionally substituted by 1 or 2 methyl groups;
R² is H, C₁₋₆alkyl, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆alkyl, C₁₋₆hetereoalkyl, or C₁₋₆alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring;
L is a bond, —CH₂(CO)—, —C₁₋₃alkylene-, —SO₂—, —C(O)—, —C(S)—, —C(NH)—, —C(O)NH—, —C(O)NHCH₂—, —C(O)N—, —C(O)OCH₂—, —C(O)O—, —C(O)C(O)—, —SO₂—NH—, or —CH₂C(O)—;
R³ is H, CN, C₁₋₆alkyl, C₅₋₁₄aryl, CH₂C₅₋₁₄aryl, CH₂C₃₋₇cycloalkyl, C₃₋₇cycloalkyl, C₃₋₇spirocycloalkyl, C₃₋₇cycloalkenyl, C₃₋₉heterocycle, or C₅₋₉heteroaryl, oxo, or R³ may join together with one R⁶ to form a fused 5-7 membered ring, and wherein each R³ group is optionally substituted by one to four substituents selected from halo, oxo, C₁₋₆alkyl, C₃₋₇cycloalkyl, C₁₋₃ fluoroalkyl, —OC₁₋₆alkyl, —C(O)R⁴, —C(O)NR⁴, —C(O)NHR⁴, C₅₋₁₄aryl, C₁₋₆hetereoalkyl, —B(OH)₂, C₃₋₉heterocycle, C₅₋₉heteroaryl, —C(O)OC₁₋₆alkyl, or two substituents may bond together to form a fused, spiro, or bridged ring and that fused, spiro, or bridged ring may optionally be substituted with R⁴;
R⁴ is CN, halo, —OC₁₋₆alkyl, C₁₋₆alkyl, C₃₋₇cycloalkyl, C₃₋₉heterocycle, or C₅₋₁₄aryl;
each R⁵ is independently H, C₁₋₃alkyl, C₃₋₆cycloalkyl, CH₂F, CHF₂, or CF₃;
each R⁶ is independently H, or C₁₋₃alkyl, C₅₋₁₄aryl, C₃₋₉heterocycle, C₅₋₉heteroaryl, —C(O)NR⁴, or —C(O)NHR⁴, or both R⁶ may together comprise 2-4 carbon atoms and join together to form a bridged ring system;
and wherein each heterocycle, heteroaryl, heteroalkyl, and heteroalkylene comprises one to three heteroatoms selected from S, N, B, or O.

2. A compound or salt according to claim 1 wherein R¹ is C₁₋₆alkyl.

3. A compound or salt according to claim 1 or claim 2 wherein X is O.

4. A compound or salt according to claim 1 wherein each R⁶ is H.

5. A compound or salt according to claim 1 wherein R² is optionally substituted phenyl.

6. A compound or salt according to claim 5 wherein R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

7. A compound or salt according to claim 1 wherein R³ is C₁₋₆alkyl, phenyl, naphthyl, cyclopentyl, cyclohexyl, pyridyl, or tetrahydropyranyl, each of which is optionally substituted by 1-3 substituents selected from halogen, C₁₋₆alkyl, —OC₁₋₆alky, C₁₋₃fluoroalkyl, or phenyl.

8. A compound or salt according to claim 1 wherein each R⁵ is methyl.

9. A compound or salt according to claim 1 wherein the stereochemistry on the carbon to which XR¹ is bound is as depicted below.

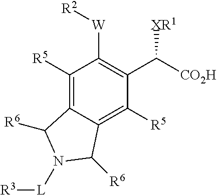

10. (canceled)

11. A pharmaceutical composition comprising a compound or salt according to claim 1.

12. A method for treating a viral infection in a patient mediated at least in part by a virus in the retrovirus family of viruses, comprising administering to said patient a composition according to claim 11.

13. The method of claim 12 wherein said viral infection is mediated by the HIV virus.

14-16. (canceled)
